(19) United States
(12) Patent Application Publication
(10) Pub. No.: US 2026/0262681 A1
(43) Pub. Date: Sep. 10, 2026

(54) PROCESS FOR MAKING ARTIFICIAL CASEIN MICELLES

(71) Applicant: FORMO FOODS GMBH, Berlin (DE)

(72) Inventors: Laurens Jans Antuma, Wageningen (NL); Julia Katharina Keppler, Wageningen (NL); Remko Marcel Boom, Wageningen (NL); Thomas Schubert, Berlin (DE)

(21) Appl. No.: 19/491,413

(22) PCT Filed: Jun. 14, 2024
(86) PCT No.: PCT/IB2024/055821

§ 371 Date: Dec. 9, 2025

(30) Foreign Application Priority Data
Jun. 14, 2023 (AU) 2023901874
Oct. 18, 2023 (AU) 2023903319

(51) Int. Cl. A23J 3/10; C07K 14/47

(52) U.S. Cl. CPC A23J 3/10; C07K 14/4732

(57) ABSTRACT

A process for the preparation of Artificial Casein Micelles (ACMs), comprising concentration of a dilute solution comprising non-micellar caseins and calcium phosphate, wherein the process is suitable for large scale and/or industrial and/or continuous operation.

### TECHNICAL FIELD

The technology described herein resides in the field of protein-based food products and dairy substitutes. More specifically, the technology relates to processes for the production of artificial protein compositions comprising protein components derived from milk, or protein components that are identical to, or homologous to those derived from milk.

### BACKGROUND ART

The following discussion of the background art is intended to facilitate an understanding of the present invention only. The discussion is not an acknowledgement or admission that any of the material referred to is or was part of the common general knowledge as at the priority date of the application.

With recent technological advancements, the production of recombinant food proteins such as caseins is becoming a feasible and sustainable alternative to conventional dairy production.

As of 5 Jul. 2022, the global dairy market was valued at approximately 830 billion U.S. dollars, and it was predicted to grow to about 1130 billion U.S. dollars by 2026. Bovine milk holds the most significant share of this market, whilst plant-derived dairy alternatives, lactose-intolerant milk, milk products that are reduced in carbohydrates and enriched in proteins, and other modified milk products such as A1 beta-casein free milk (A2 milk) are increasingly prominent in an increasingly informed market of diet-conscious consumers.

Increasing awareness for sustainability and animal welfare draws a growing number of people to follow vegetarian or vegan diets. Consequently, the alternative protein industry is one of the fastest growing industries with a global market expected to grow fivefold by 2030. Many food manufacturers have developed plant-based alternatives to meet the increased consumer demand. However, plant-based dairy, and specifically plant-based cheese alternatives, often fail to meet consumer expectations with regards to sensory and nutritional qualities.

Mammalian-derived milk is a highly complex liquid composition comprising, aside from water, thousands of different compounds, from lipids, triglycerides, carbohydrates, saccharides, peptides, inorganic salts and other molecular entities. Although many consider mammalian-derived milk, including bovine milk, to be an ideal nutrition source, various alternatives to mammalian-derived milk are now successfully on the market, including plant- or nut-based milks, such as soy, almond, or coconut milk, and are accepted by consumers for reasons related to mammalian-derived milk's allergenicity, lactose intolerance of certain components, personal preference, or the perception of adverse environmental impacts arising from the dairy industry.

For example, the majority of mammalian-derived milk is sourced from ruminant animals including cows, buffalos, yaks, goats and sheep, as well as pseudo-ruminants such as camels, alpacas and llamas. Cattle-rearing and ruminant livestock agriculture in general produces more global warming greenhouse gases, as measured in carbon dioxide (CO₂) equivalents, than transportation, according to a recent UN assessment. Ruminants are estimated to account for 10% of total greenhouse gas emissions in Australia. Ruminants produce methane (CH₄) as a by-product of digestion via anaerobic microbial feed fermentation in the rumen and, to a lesser extent, the large intestine. This process is referred to as methanogenesis.

Methane absorbs solar infrared radiation efficiently, and it has a global warming potential 25 times that of CO₂. The ruminal microbial population is made up of bacteria, protozoa, fungi, and bacteriophages, all of which work together to digest ingested organic matter and produce CO₂, H₂, volatile fatty acids, and formates. These end-products are used by methanogenic archaea in the rumen, which produces CH₄. Although the generation of CH₄ lowers the partial pressure of H₂, this has the potential to cause problems as it also limits the amount of energy and carbon available for the synthesis of volatile fatty acids, which are critical for ruminant nutrition and could otherwise restrict rumen fermentation. The majority of CH₄ generated by ruminants is exhaled or discharged via the mouth, resulting in a waste of up to 12% of gross caloric intake in the ruminant diet. In addition, producing a single glass of dairy milk from cows consumes up to nine times more land, and significantly more water, than any of the plant-derived milk alternatives.

Attempts to address these environmental issues with plant-derived milks including soy, almond, or coconut milk, for example, fall short in both flavour and utility. In addition, a major portion of dairy milk's industrial and cultural value originates from its use in derivative goods such as cheese, yoghurt, cream, or butter. While dairy substitute plant-derived milks address some environmental and health problems (and provide sufficient flavour for a minor portion of the consumer population), when exposed to the same procedures as dairy milk, they virtually always fail to generate such derivative goods.

There is a need then, for an alternative dairy substitute or composition with desirable flavour and performance characteristics, such as a composition that replicates dairy flavours, whilst minimising foodborne pathogens, and that potentially has a lower environmental impact in production, while retaining the ability to be used for derivative or downstream applications of dairy milk and providing a nutritional profile similar to that of mammalian-derived milk.

The protein content of bovine milk required for most derivative products such as cheese is primarily comprised of four distinct caseins; αₛ₁-casein, αₛ₂-casein, β-casein and κ-casein. Cheese is the third most unsustainable animal product in the world (in terms of greenhouse gas emissions per kg of product), yet plant-based alternatives released onto the market in the previous decade have not decreased demand of dairy cheese. On the contrary, consumption of mozzarella cheese in the United States and other developing countries is increasing year after year. Due to a lack of casein proteins, current cheese replacements do not match the functionality (including melt behaviour and browning behaviour when cooked or grilled), texture, nutrition, and taste of dairy cheese. Meanwhile, human allergies to milk products are most often caused by the αₛ₁-casein protein present in dairy milk.

Cheese is produced in a process whereby whey proteins are separated from dairy milk, leaving behind a suspension of casein micelles comprising αₛ₁-casein, αₛ₂-casein, β-casein and κ-casein. This suspension of casein micelles is then subjected to coagulation and treatment with rennet enzymes to form a curd, which is then aged to form cheeses of various types. Achieving a desirable texture, hardness, elasticity and other functional properties such as melt behaviour in the cheese and desirable browning behaviour when cooked or grilled, is highly dependent on micelle size and mineral content of the micelles in the suspension of casein micelles used to generate the curd. In general terms, if the micelles are not large enough, they tend not to form sufficiently firm curds when subjected to coagulation and rennetisation, resulting in undesirable texture, hardness, elasticity and other functional properties in the downstream products arising from coagulation and rennetisation. Furthermore, when the micelles are not large enough, they tend to be unable to entrap sufficient salts within their micellar structure to impart good flavour to the downstream products arising from coagulation and rennetisation.

One issue that many producers in the field of dairy substitutes have in common is the challenge in scaling at a rapid and cost-effective rate. Precision fermentation is a technology that has the potential to supply consumers with animal-free cheese products, that are of similar nutritional and sensorial quality as cheese made from bovine milk. With this technique, milk proteins, such as caseins, can be produced by genetically engineering microbial hosts (e.g. yeasts or bacteria). Precision fermentation promises to require less land, water and energy resources as microorganisms can be grown in large quantities in bioreactors. Decreasing production costs and recent advances in technology have made the mass production of these so-called recombinant food proteins become more realistic than ever.

Since the development and production of recombinant caseins can be costly and difficult, it would be desirable to produce artificial casein micelles, having similar structural and functional properties, including in terms of mineral content and micelle size, to those observed in dairy milk, but without necessarily requiring the presence of all four caseins present in dairy milk (αₛ₁-casein, αₛ₂-casein, β-casein and κ-casein), thereby greatly simplifying the production of such artificial casein micelles, especially where the casein proteins used are sourced from non-dairy origins such as via recombinant microorganisms.

One challenge in the use of recombinant caseins for cheese production is the assembly of the individual recombinant caseins into casein micelles, required as a precursor to coagulation and/or rennetisation to form the necessary curds for cheese production. The re-assembly of casein micelles with non-micellar bovine caseins, creating so-called artificial casein micelles (ACM), has been achieved in vitro by progressively mixing salt solutions with a casein solution to form calcium phosphate nanoclusters, which initiates the formation of ACMs (Schmidt, D. G., Koops, J., & Westerbeek, D. (1977). Properties of artificial casein micelles. 1. Preparation, size distribution and composition. Netherlands Milk and Dairy Journal, 31(4), 328-341).¹ The method developed by Schmidt involves utilising an array of pumps to mix a casein solution and three separate salt solutions by pumping them gradually into a central vessel that contains an initial amount of water over a time period of an hour.

Such methods for the production of produce artificial casein micelles (ACMs) are difficult to scale up and/or to speed up, and typically result in micelles of impaired functionality when produced at a larger scale due to uncontrolled calcium phosphate crystallization (Antuma et al., 2024),⁹ especially when applied to large batches.

There is a need to develop alternative processes to achieve more controlled calcium phosphate supersaturation and formation of artificial casein micelles (ACMs), amenable to the re-assembly of recombinant caseins into ACMs on an industrial scale.

It is against this background that the present invention has been developed.

### SUMMARY OF THE INVENTION

By developing a process to (re) assemble non-micellar caseins into casein micelles, the present inventors have created artificial casein micelles (ACMs) with properties very similar to natural bovine casein micelles in terms of micelle size and mineral content. These artificial micelles may also be coagulated by the action of rennet to create the same cheese textures as those produced from bovine milk, allowing for the complete replacement of the functionality of bovine casein micelles.

In one embodiment, the disclosure herein provides a process for the preparation of Artificial Casein Micelles (ACMs), comprising;
a) preparing a solution, comprising non-micellar caseins and calcium phosphate, wherein the concentration of calcium phosphate is dilute; and
b) concentrating the solution comprising non-micellar caseins and calcium phosphate to form a solution comprising ACMs;
   wherein the formation of the ACMs is induced by step b); concentrating the solution comprising non-micellar caseins and calcium phosphate, without the addition of any further salts or caseins to the solution prepared in step a).

In some embodiments, step b) of concentrating the solution comprising non-micellar caseins and calcium phosphate is performed via removal of solvent via evaporation under reduced pressure, or removal of solvent via membrane processes, or removal of solvent via forward osmosis, or removal of solvent via reverse osmosis.

In some embodiments, step b) of concentrating the solution comprising non-micellar caseins further comprises controlling the pH of the solution while concentrating the solution, via addition of a suitable base, to maintain the pH of the solution at a pH falling within the range of pH 5 to pH 7.5; preferably within the range of pH 6 to pH 7; most preferably at a pH of 6.7; optionally wherein the base is sodium hydroxide.

In some embodiments, the solution prepared in step a) further comprises one or more additional species selected from the group consisting of; calcium, magnesium, sodium, potassium, chloride, phosphate, phosphorus, citrate, carbonate, sulfate, nitrate, hydroxide, and lactate.

In some embodiments, the solution comprising ACMs formed in step b) has a pH falling within the range of pH 5 to pH 7.5; preferably wherein the solution comprising ACMs formed in step b) has a pH falling within the range of pH 6 to pH 7; most preferably the solution comprising ACMs formed in step b) has a pH of 6.7.

In some embodiments, the solution prepared in step a) has a pH, prior to commencement of step b), falling within the range of pH 6 to pH 8; preferably wherein the solution prepared in step a) has a pH, prior to commencement of step b), falling within the range of pH 6.2 to pH 7.8; most preferably wherein the solution prepared in step a) has a pH, prior to commencement of step b), falling within the range of pH 6.5 to 7.5.

In some embodiments, the non-micellar caseins are one or more non-micellar caseins selected from the group consisting of; αₛ₁-casein, αₛ₂-casein, β-casein and κ-casein.

In some embodiments, the non-micellar caseins are isolated from mammalian milk, or wherein the non-micellar caseins are recombinantly produced non-micellar caseins.

In some embodiments, the non-micellar caseins are phosphorylated or not phosphorylated; and/or wherein the non-micellar caseins are glycosylated or not glycosylated.

In some embodiments, the solution comprising ACMs formed in step b) comprises;
calcium, at a concentration falling within the range of 20 mmol/kg to 40 mmol/kg; preferably falling within the range of 25 mmol/kg to 35 mmol/kg; most preferably falling within the range of 26 mmol/kg to 31 mmol/kg; and/or
magnesium, at a concentration falling within the range of 2 mmol/kg to 8 mmol/kg; preferably falling within the range of 4 mmol/kg to 6 mmol/kg; and/or
inorganic phosphate, at a concentration falling within the range of 10 mmol/kg to 30 mmol/kg; preferably falling within the range of 15 mmol/kg to 25 mmol/kg; most preferably falling within the range of 19 mmol/kg to 23 mmol/kg; and/or
total phosphorus, at a concentration falling within the range of 20 mmol/kg to 40 mmol/kg; preferably falling within the range of 25 mmol/kg to 35 mmol/kg; most preferably falling within the range of 26 mmol/kg to 32 mmol/kg; and/or
citrate, at a concentration falling within the range of 2 mmol/kg to 20 mmol/kg; preferably falling within the range of 7 mmol/kg to 11 mmol/kg; and/or
sodium, at a concentration falling within the range of 5 mmol/kg to 100 mmol/kg; preferably falling within the range of 10 mmol/kg to 80; most preferably falling within the range of 15 mmol/kg to 75 mmol/kg; and/or
potassium, at a concentration falling within the range of 10 mmol/kg to 50 mmol/kg; preferably falling within the range of 15 mmol/kg to 40 mmol/kg; and/or
chloride, at a concentration falling within the range of 5 mmol/kg to 100 mmol/kg; preferably falling within the range of 10 mmol/kg to 80 mmol/kg; most preferably falling within the range of 15 mmol/kg to 75 mmol/kg.

In some embodiments, the solution prepared in step a) comprises Mg²⁺, PO₄³⁻ Ca²⁺, citrate and non-micellar caseins in the ratios in which they occur in bovine milk.

In some embodiments, the solution comprising ACMs formed in step b) comprises total caseins at a concentration falling within the range of 15 g/L to 100 g/L; preferably falling within the range of 20 g/L to 30 g/L.

In some embodiments, the solution comprising ACMs formed in step b) comprises ACMs with a Z-average diameter falling within the range of 40 to 500 nm.

In some embodiments, the solution comprising ACMs formed in step b) comprises ACMs with hydration values (g water/g micellar casein) falling within the range of 2 to 4.

In some embodiments, the process of the present invention further comprises the step of;
c) coagulating the solution comprising ACMs formed in step b), to form a curds composition.

In some embodiments, step c) of coagulating the solution comprising ACMs formed in step b), to form a curds composition, is achieved via addition of an acid, and/or microbial acidification, and/or addition of a renneting agent.

In some embodiments, the curds composition has a Maximum G′ (storage modulus) falling within the range of 50 Pa to 400 Pa, preferably after 1 hour of incubation with a renneting agent.

In some embodiments, the process of the present invention further comprises the step of;
(d) aging and/or maturing the curds composition, to form a cheese composition.

In some embodiments, the process does not comprise any animal-derived protein.

In some embodiments, the process is conducted on an industrial scale; and/or wherein the process is conducted continuously; and/or wherein the process is conducted on a scale capable of producing 100 L to 10,000 L of the solution comprising ACMs formed in step b) in a single batch, or process run.

In some embodiments, the solution prepared in step a) is prepared with a Concentration factor falling within the range of 3× to 50×; preferably wherein the solution prepared in step a) is prepared with a Concentration factor falling within the range of 6× to 30×.

In some embodiments, step b) of concentrating the solution comprising non-micellar caseins and calcium phosphate is performed via removal of solvent via evaporation under reduced pressure, at a reduced pressure falling within the range of 10 mbar to 300 mbar, corresponding to a boiling point of water falling within the range of 7° C. to 70° C.

In some embodiments, the solution prepared in step a) comprising a dilute concentration of calcium phosphate has;
calcium at a concentration falling within the range of 0.6 mM to 10 mM; and/or
phosphate at a concentration falling within the range of 0.4 mM to 7.3 mM.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a plot of evaporation rate as a function of heating temperature during vacuum evaporation at 62 mBar.

FIG. 2 is a plot of the Diameter (Z-average) of artificial casein micelles (Series 1 VE-ACMs) prepared with; a) increasing concentration factors; and b) increasing evaporation rates.

FIG. 3 is a plot of the Diameter (Z-average) of artificial casein micelles prepared via vacuum evaporation (Series 2 VE-ACMs) prepared with increasing preparation time (corresponding to decreasing evaporation rates, with concentration factor held constant at 6×), compared to S-ACM produced with the method of Schmidt¹ with a preparation time of 60 minutes, ACMs prepared via forward osmosis (FO-ACMs) produced with preparation times of 28, 38 and 60 minutes, and ACM prepared via reverse osmosis (RO-ACM) produced with a preparation time of 22 hours.

FIG. 4 is a Scanning Electron Microscopy (SEM) image of bovine skim milk, taken at 100,000× magnification. Scale bar=500 nm.

FIG. 5 is a series of Scanning Electron Microscopy (SEM) images of; (a) S-ACM produced with the method of Schmidt¹; and (b) VE-ACM (Series 2) produced in accordance with the process of the present invention via vacuum evaporation; both with a preparation time of 1 hour; and (c) FO-ACM produced in accordance with the process of the present invention via forward osmosis with a preparation time of 1 hour; and (d) RO-ACM produced in accordance with the process of the present invention via reverse osmosis with a preparation time of 22 hours. SEM pictures were taken at 100,000× magnification. Scale bar=500 nm.

FIG. 6 are plots of the Micellar casein of the prepared samples of VE-ACM (Series 1) as a percentage of the total casein for VE-ACMs prepared with; a) increasing concentration factors; and b) with increasing evaporation rates.

FIG. 7 are plots of the Maximum G′ (storage modulus; measure of the firmness) of the produced curds upon rennet-induced coagulation of the Series 1 VE-ACM samples prepared with; a) increasing concentration factors; and b) different evaporation rates. The dotted lines represent the maximum storage modulus of bovine skim milk (lower dotted line) and S-ACM produced by the method of Schmidt¹ (upper dotted line).

FIG. 8 is a plot of the development of the storage modulus G′ over an hour of incubation with rennet at 30° C. of the Series 2 vacuum evaporation ACM sample prepared with a heating temperature of 63° C. (VE-ACM), the ACM prepared via forward osmosis in a 60 minute time period (FO-ACM), and the ACM prepared via reverse osmosis in a 22 hour time period (RO-ACM), compared with that of ACM prepared by the method of Schmidt¹ (S-ACM).

### DEFINITIONS

Throughout this specification, unless the context requires otherwise, the word “comprise” or variations such as “comprises” or “comprising”, will be understood to imply the inclusion of a stated integer or group of integers but not the exclusion of any other integer or group of integers.

Unless expressly indicated as otherwise, the term “%” shall be understood throughout this specification as referring to weight %, or wt. %.

As used herein, the term “micelle”, and grammatical variations thereof, shall be understood to mean a generally (or roughly) spherical supramolecular structure that exists as a dispersion within a composition or solution. A micelle can have, e.g., a surface that is composed of a charged outer layer. A micelle can encapsulate one or more biomolecules. For example, a micelle can encapsulate two or more proteins (e.g., a β-casein protein and a κ-casein protein). A micelle can have diameter of between about 10 nm and about 500 nm. Additional aspects and characteristics of micelles are known in the art.

As used herein, the term “Artificial Casein Micelle” shall be used interchangeably with the acronym “ACM”, and the plural equivalents “Artificial Casein Micelles” and “ACMs”.

As used herein, the terms “about,” “approximately,” and grammatical variations thereof, shall be understood to mean within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

As used herein, the term “non-micellar casein” shall be understood to mean any form of casein that is not part of a micelle structure, including any form of casein isolated from any source, including αₛ₁-casein, αₛ₂-casein, β-casein and κ-casein isolates of any mammalian species, as well as any synthetically or recombinantly produced αₛ₁-casein, αₛ₂-casein, β-casein or κ-casein, and including variants having at least 80% sequence homology with any mammalian αₛ₁-casein, αₛ₂-casein, β-casein or κ-casein sequence, and including such variants having at least 80% sequence homology with any mammalian αₛ₁-casein, αₛ₂-casein, β-casein or κ-casein sequence with or without post-translational modifications such as glycosylation and/or phosphorylation. Some embodiments of the artificial micelles of the present invention comprise synthetically or recombinantly produced αₛ₁-casein, αₛ₂-casein, β-casein or κ-casein variants possessing a sequence homology with any mammalian αₛ₁-casein, αₛ₂-casein, β-casein or κ-casein sequence, wherein the sequence homology with any mammalian αₛ₁-casein, αₛ₂-casein, β-casein or κ-casein sequence is selected from the group of sequence homologies consisting of; 80%, 81%, 82%, 83%, 84%, 85%, 86%, 87%, 88%, 89%, 90%, 91%, 92%, 93%, 94%, 95%, 96%, 97%, 98%, 99%, and 100% sequence homology with any mammalian αₛ₁-casein, αₛ₂-casein, β-casein or κ-casein sequence, with or without post-translational modifications such as glycosylation and/or phosphorylation. It should also be understood that any of the aforementioned caseins include salts of said caseins.

As used herein, the term “animal-derived protein” shall be understood to mean any protein derived from any mammalian animal source. It shall further be understood that the term “animal-derived protein” does not include any proteins that are synthetically or recombinantly produced.

As used herein, the term “Concentration factor” shall be understood to refer to the ratio of the initial volume of the precursor solution comprising non-micellar caseins and calcium phosphate, to the final volume of the concentrated solution comprising ACMs. In other words, “Concentration factor” is calculated by dividing the initial volume of the precursor solution comprising non-micellar caseins and calcium phosphate, by the final volume of the concentrated solution comprising ACMs. Thus, for example, where the initial volume of the precursor solution comprising non-micellar caseins and calcium phosphate is 900 ml and the final volume of the concentrated solution comprising ACMs is 150 ml, the concentration factor will be 900/150=6×.

Applying the term “Concentration factor” to the process of the present invention, in which the key steps involve; a) preparing a solution, comprising non-micellar caseins and calcium phosphate, wherein the concentration of calcium phosphate is dilute; and b) concentrating the solution comprising non-micellar caseins and calcium phosphate to form a solution comprising ACMs; it should be understood that the initial solution prepared in step a) will contain the target mineral concentrations of the final solution produced in step b), divided by the concentration factor. Therefore, where it is referred to herein that the solution prepared in step a) is “6×” it should be understood that the resulting solution produced from step b) will be concentrated 6 times with respect to the solution prepared in step a). Thus, the solutions prepared in step a) are prepared with the target concentrations of the resulting solution produced from step b) in mind.

As used herein, the phrase “wherein the concentration of calcium phosphate is dilute” as it applies to key step a) of the process of the present invention, shall be understood to mean that the concentration of calcium phosphate is sufficiently low enough to avoid the spontaneous assembly of casein micelles, prior to process step b) of concentrating the solution comprising non-micellar caseins and calcium phosphate to form a solution comprising ACMs. Thus, it shall be understood that in accordance with the processes of the present invention, the solutions prepared in step a) will not contain any casein micelles, by virtue of the fact that the concentration of calcium phosphate in the solutions prepared in step a) is sufficiently low that all of the calcium phosphate present, is completely solubilised, thereby preventing the spontaneous formation of any colloidal structures such as micelles, and that micelle formation only occurs during the concentration step b), once the solution of step b) becomes sufficiently concentrated in calcium phosphate for precipitation of the calcium phosphate to begin to occur, and thereby initiate the formation of ACMs.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DETAILED DESCRIPTION

The disclosure herein provides an unprecedented protocol that allows for the preparation of ACM solutions comprising αₛ₁-casein and/or αₛ₂-casein and/or β-casein and/or κ-casein, from dilute solutions of non-micellar caseins, in a highly controlled manner that is suitable for large scale or industrial scale production, including continuous production.

Advantageously, the ACM solutions of the present invention possess suitable micelle sizes and micellar mineral salt content for applicability to downstream product manufacturing such as cheesemaking.

This protocol is directly applicable to the preparation of artificial casein micelles from recombinantly produced αₛ₁-casein and/or αₛ₂-casein and/or β-casein and/or κ-casein, whether or not the aforementioned caseins are glycosylated and/or phosphorylated.

Without wishing to be bound by theory, the present inventors believe that crystallization of calcium phosphate in a solution comprising non-micellar caseins, initiates the formation of artificial casein micelles (ACMs), and that the preparation of ACMs in accordance with the process of the present invention therefore involves the preparation of a non-micellar casein precursor solution in which the concentration of calcium phosphate is sufficiently dilute to avoid excessive calcium phosphate crystallization, and thus the spontaneous formation of ACMs, prior to the concentration of the precursor solution via any suitable process such as, but not limited to, evaporation or membrane processes.

Accordingly, the disclosure herein provides and enables a process for the preparation of Artificial Casein Micelles (ACMs), comprising;
(a) preparing a solution, comprising non-micellar caseins and calcium phosphate, wherein the concentration of calcium phosphate is dilute; and
(b) concentrating the solution comprising non-micellar caseins and calcium phosphate to form a solution comprising ACMs;
wherein the formation of the ACMs is induced by step b); concentrating the solution comprising non-micellar caseins and calcium phosphate, without the addition of any further salts or caseins to the solution prepared in step a).

Previously, non-micellar caseins have been successfully re-assembled into artificial casein micelles (ACM) by slowly mixing sodium caseinate with salt solutions to the concentrations found in milk (Schmidt et al., 19771).¹ The progressive addition of salts leads to supersaturation of calcium and phosphate, which cluster and consequently form Calcium Phosphate (CaP) nuclei with which the caseins can interact, and thereby spontaneously assemble into the micelle structures that are necessary for the downstream production of curd-derived products including cheeses. However, these approaches are not amenable to large scale production due to the tendency of localised high concentrations of calcium and phosphate to occur when concentrated salt solutions are added to large batches. The process of the present invention obviates these limitations.

To provide proof of principle, artificial casein micelles were prepared in accordance with the process of the present invention, from bovine sodium caseinate. Artificial casein micelles (ACM) were prepared by concentrating a dilute solution containing Ca²⁺, Mg²⁺, PO₄³⁻, C₆H₅O₇³⁻ and caseinate ions in the ratios in which they occur in bovine milk, via vacuum evaporation at a pressure of 62 to 63 mBar (corresponding to a boiling point for water of about 37° C.), and using a water bath temperature (heating temperature) falling within the range of 46° C. to 80° C., to vary the rate of concentration.

The person skilled in the art will appreciate that whilst the abovementioned preferred conditions were utilized to provide proof of principle of the present protocols, variations may be made to each of these preferred conditions without departing from the general principle of application provided by the disclosure of the core inventive concept set forth herein.

For example, the water bath (heating bath) temperature may be varied within any suitable range above or below the preferred temperature range of 46° C. to 80° C., and the vacuum pressure applied may also be varied (to correspond to a different boiling temperature for water).

Without departing from the scope of the present invention, the water bath temperature may be set at any temperature selected from the group consisting of; 15±0.5° C., 16±0.5° C., 17±0.5° C., 18±0.5° C., 19±0.5° C., 20±0.5° C., 21±0.5° C., 22±0.5° C., 23±0.5° C., 24±0.5° C., 25±0.5° C., 26±0.5° C., 27±0.5° C., 28±0.5° C., 29±0.5° C., 30±0.5° C., 31±0.5° C., 32±0.5° C., 33±0.5° C., 34±0.5° C., 35±0.5° C., 36±0.5° C., 37±0.5° C., 38±0.5° C., 39±0.5° C., 40±0.5° C., 41±0.5° C., 42±0.5° C., 43±0.5° C., 44±0.5° C., 45±0.5° C., 46±0.5° C., 47±0.5° C., 48±0.5° C., 49±0.5° C., 50±0.5° C., 51±0.5° C., 52±0.5° C., 53±0.5° C., 54±0.5° C., 55±0.5° C., 56±0.5° C., 57±0.5° C., 58±0.5° C., 59±0.5° C., 60±0.5° C., 61±0.5° C., 62±0.5° C., 63±0.5° C., 64±0.5° C., 65±0.5° C., 66±0.5° C., 67±0.5° C., 68±0.5° C., 69±0.5° C., 70±0.5° C., 71±0.5° C., 72±0.5° C., 73±0.5° C., 74±0.5° C., 75±0.5° C., 76±0.5° C., 77±0.5° C., 78±0.5° C., 79±0.5° C., 80±0.5° C., 81±0.5° C., 82±0.5° C., 83±0.5° C., 84±0.5° C., 85±0.5° C., 86±0.5° C., 87±0.5° C., 88±0.5° C., 89±0.5° C., 90±0.5° C., 91±0.5° C., 92±0.5° C., 93±0.5° C., 94±0.5° C., 95±0.5° C., 96±0.5° C., 97±0.5° C., 98±0.5° C., 99±0.5° C. and 100±0.5° C.

Similarly, the removal of solvent via evaporation under reduced pressure, may be performed at a reduced pressure falling within the range of 10 mbar to 300 mbar, for example, the removal of solvent via evaporation under reduced pressure, may be performed at a reduced pressure selected from the group consisting of 10 mbar, 11 mbar, 12 mbar, 13 mbar, 14 mbar, 15 mbar, 16 mbar, 17 mbar, 18 mbar, 19 mbar, 20 mbar, 21 mbar, 22 mbar, 23 mbar, 24 mbar, 25 mbar, 26 mbar, 27 mbar, 28 mbar, 29 mbar, 30 mbar, 31 mbar, 32 mbar, 33 mbar, 34 mbar, 35 mbar, 36 mbar, 37 mbar, 38 mbar, 39 mbar, 40 mbar, 41 mbar, 42 mbar, 43 mbar, 44 mbar, 45 mbar, 46 mbar, 47 mbar, 48 mbar, 49 mbar, 50 mbar, 51 mbar, 52 mbar, 53 mbar, 54 mbar, 55 mbar, 56 mbar, 57 mbar, 58 mbar, 59 mbar, 60 mbar, 61 mbar, 62 mbar, 63 mbar, 64 mbar, 65 mbar, 66 mbar, 67 mbar, 68 mbar, 69 mbar, 70 mbar, 71 mbar, 72 mbar, 73 mbar, 74 mbar, 75 mbar, 76 mbar, 77 mbar, 78 mbar, 79 mbar, 80 mbar, 81 mbar, 82 mbar, 83 mbar, 84 mbar, 85 mbar, 86 mbar, 87 mbar, 88 mbar, 89 mbar, 90 mbar, 91 mbar, 92 mbar, 93 mbar, 94 mbar, 95 mbar, 96 mbar, 97 mbar, 98 mbar, 99 mbar, 100 mbar, 101 mbar, 102 mbar, 103 mbar, 104 mbar, 105 mbar, 106 mbar, 107 mbar, 108 mbar, 109 mbar, 110 mbar, 111 mbar, 112 mbar, 113 mbar, 114 mbar, 115 mbar, 116 mbar, 117 mbar, 118 mbar, 119 mbar, 120 mbar, 121 mbar, 122 mbar, 123 mbar, 124 mbar, 125 mbar, 126 mbar, 127 mbar, 128 mbar, 129 mbar, 130 mbar, 131 mbar, 132 mbar, 133 mbar, 134 mbar, 135 mbar, 136 mbar, 137 mbar, 138 mbar, 139 mbar, 140 mbar, 141 mbar, 142 mbar, 143 mbar, 144 mbar, 145 mbar, 146 mbar, 147 mbar, 148 mbar, 149 mbar, 150 mbar, 151 mbar, 152 mbar, 153 mbar, 154 mbar, 155 mbar, 156 mbar, 157 mbar, 158 mbar, 159 mbar, 160 mbar, 161 mbar, 162 mbar, 163 mbar, 164 mbar, 165 mbar, 166 mbar, 167 mbar, 168 mbar, 169 mbar, 170 mbar, 171 mbar, 172 mbar, 173 mbar, 174 mbar, 175 mbar, 176 mbar, 177 mbar, 178 mbar, 179 mbar, 180 mbar, 181 mbar, 182 mbar, 183 mbar, 184 mbar, 185 mbar, 186 mbar, 187 mbar, 188 mbar, 189 mbar, 190 mbar, 191 mbar, 192 mbar, 193 mbar, 194 mbar, 195 mbar, 196 mbar, 197 mbar, 198 mbar, 199 mbar, 200 mbar, 201 mbar, 202 mbar, 203 mbar, 204 mbar, 205 mbar, 206 mbar, 207 mbar, 208 mbar, 209 mbar, 210 mbar, 211 mbar, 212 mbar, 213 mbar, 214 mbar, 215 mbar, 216 mbar, 217 mbar, 218 mbar, 219 mbar, 220 mbar, 221 mbar, 222 mbar, 223 mbar, 224 mbar, 225 mbar, 226 mbar, 227 mbar, 228 mbar, 229 mbar, 230 mbar, 231 mbar, 232 mbar, 233 mbar, 234 mbar, 235 mbar, 236 mbar, 237 mbar, 238 mbar, 239 mbar, 240 mbar, 241 mbar, 242 mbar, 243 mbar, 244 mbar, 245 mbar, 246 mbar, 247 mbar, 248 mbar, 249 mbar, 250 mbar, 251 mbar, 252 mbar, 253 mbar, 254 mbar, 255 mbar, 256 mbar, 257 mbar, 258 mbar, 259 mbar, 260 mbar, 261 mbar, 262 mbar, 263 mbar, 264 mbar, 265 mbar, 266 mbar, 267 mbar, 268 mbar, 269 mbar, 270 mbar, 271 mbar, 272 mbar, 273 mbar, 274 mbar, 275 mbar, 276 mbar, 277 mbar, 278 mbar, 279 mbar, 280 mbar, 281 mbar, 282 mbar, 283 mbar, 284 mbar, 285 mbar, 286 mbar, 287 mbar, 288 mbar, 289 mbar, 290 mbar, 291 mbar, 292 mbar, 293 mbar, 294 mbar, 295 mbar, 296 mbar, 297 mbar, 298 mbar, 299 mbar and 300 mbar; and/or a boiling point of water selected from the group consisting of 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. and 70° C.

Whilst proof of principle of the process of the present invention was provided by preparing a solution in step a) comprising Mg²⁺, PO₄³⁻ Ca²⁺, citrate and non-micellar caseins in the ratios in which they occur in bovine milk, the skilled addressee will understand that the present invention may also be implemented to produce ACMs with utility in food production in accordance with the present invention by preparing a solution in step a) comprising Mg²⁺, PO₄³⁻ Ca²⁺, citrate and non-micellar caseins in ratios that do not correspond to those in which they occur in bovine milk.

Furthermore, the preferred salts utilized in the exemplary embodiments described herein may be varied via the use of alternative salts to provide the required or desired sources of Ca²⁺, Mg²⁺, K⁺, Na⁺, PO₄³⁻, and C₆H₅O₇³⁻ ions. For example, other salts such as, without limitation; calcium acetate, calcium carbonate, calcium citrate, calcium gluconate, calcium sulfate, calcium phosphate, calcium nitrate, magnesium acetate, magnesium carbonate, magnesium citrate, magnesium gluconate, magnesium sulfate, magnesium phosphate, magnesium nitrate, potassium acetate, potassium carbonate, potassium citrate, potassium gluconate, potassium sulfate, dipotassium phosphate, tripotassium phosphate, potassium nitrate, sodium acetate, sodium carbonate, monosodium citrate, disodium citrate, sodium gluconate, sodium sulfate, monosodium phosphate, trisodium phosphate, and sodium nitrate may be used as alternative sources of Ca²⁺, Mg²⁺, K⁺, Na⁺, PO₄³⁻, and C₆H₅O₇³⁻ ions.

Additionally, the solution prepared in step a) of the process of the present invention may further comprise any source of one or more additional species selected from the group consisting of; calcium, magnesium, sodium, potassium, chloride, phosphate, phosphorus, citrate, carbonate, sulfate, nitrate, hydroxide, and lactate.

Similarly, whilst the exemplary embodiments of the present invention provide proof of the principle of general application of concentrating solutions comprising non-micellar caseins and dilute calcium phosphate to initiate micelle ACM formation via vacuum evaporation, or evaporation under reduced pressure, the person skilled in the art will understand that this principle of general application may be applied to alternative means of concentrating solutions comprising non-micellar caseins and dilute calcium phosphate to initiate micelle ACM formation, such as, but not limited to membrane processes, or removal of solvent via forward osmosis, or removal of solvent via reverse osmosis, without departing from the scope of the present invention.

In accordance with the process of the present invention, the solution prepared in step a) has a pH, prior to commencement of step b), selected from the group consisting of; pH 6, pH 6.1, pH 6.2, pH 6.3, pH 6.4, pH 6.5, pH 6.6, pH 6.7, pH 6.8, pH 6.9, pH 7, pH 7.1, pH 7.2, pH 7.3, pH 7.4, pH 7.5, pH 7.6, pH 7.7, pH 7.8, pH 7.9, and pH 8.

Although the exemplary embodiments describing proof of the principle of general application provided by the process of the present invention were designed such that the solution comprising ACMs formed in step b) had a pH falling within the range of pH 5 to pH 7.5; preferably wherein the solution comprising ACMs formed in step b) had a pH falling within the range of pH 6 to pH 7; most preferably the solution comprising ACMs formed in step b) had a pH of 6.7, the skilled addressee will appreciate that without departing from the invention described herein, the process of the present invention includes the aspect of additionally controlling and/or maintaining the pH of the solution while concentrating the solution, via addition of a suitable base, to maintain the pH of the solution at a pH falling within the range of pH 5 to pH 7.5; preferably within the range of pH 6 to pH 7; most preferably at a pH of 6.7.

Suitable bases include sodium hydroxide, potassium hydroxide, sodium carbonate, sodium bicarbonate, ammonium bicarbonate, calcium carbonate, potassium carbonate, potassium bicarbonate, or any other organic base or inorganic base that is “Generally recognized as safe” (GRAS) in accordance with the United States Food and Drug Administration (FDA) designation that a chemical or substance added to food is considered safe by experts under the conditions of its intended use.

Whether the solution comprising ACMs formed in step b) is predetermined by design of the solution prepared in step a), or controlled or maintained via addition of a suitable base during the concentration process of step b), the pH of the solution comprising ACMs formed in step b) may be selected from the group consisting of; pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, and pH 7.5, without departing from the scope of the present invention.

In accordance with some embodiments of the process of the present invention, the solution prepared in step a) is prepared with a Concentration factor (in view of the target concentrations to be achieved in the solution comprising ACMs formed in step b) of the process), selected from the group consisting of; 3×, 3.5×, 4×, 4.5×, 5×, 5.5×, 6×, 6.5×, 7×, 7.5×, 8×, 8.5×, 9×, 9.5×, 10×, 10.5×, 11×, 11.5×, 12×, 12.5×, 13×, 13.5×, 14×, 14.5×, 15×, 15.5×, 16×, 16.5×, 17×, 17.5×, 18×, 18.5×, 19×, 19.5×, 20×, 20.5×, 21×, 21.5×, 22×, 22.5×, 23×, 23.5×, 24×, 24.5×, 25×, 25.5×, 26×, 26.5×, 27×, 27.5×, 28×, 28.5×, 29×, 29.5×, 30×, 30.5×, 31×, 31.5×, 32×, 32.5×, 33×, 33.5×, 34×, 34.5×, 35×, 35.5×, 36×, 36.5×, 37×, 37.5×, 38×, 38.5×, 39×, 39.5×, 40×, 40.5×, 41×, 41.5×, 42×, 42.5×, 43×, 43.5×, 44×, 44.5×, 45×, 45.5×, 46×, 46.5×, 47×, 47.5×, 48×, 48.5×, 49×, 49.5×, and 50×.

In accordance with some embodiments of the process of the present invention, the solution prepared in step a) comprising a dilute concentration of calcium phosphate has calcium at a concentration selected from the group consisting of; 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, and 20 mM.

In accordance with some embodiments of the process of the present invention, the solution prepared in step a) comprising a dilute concentration of calcium phosphate has phosphate at a concentration selected from the group consisting of; 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, and 20 mM.

In accordance with some embodiments of the process of the present invention, the solution comprising ACMs formed in step b) of the process, may comprise calcium, at a concentration selected from the group consisting of; 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, 30 mmol/kg, 31 mmol/kg, 32 mmol/kg, 33 mmol/kg, 34 mmol/kg, 35 mmol/kg, 36 mmol/kg, 37 mmol/kg, 38 mmol/kg, 39 mmol/kg, and 40 mmol/kg.

In accordance with some embodiments of the process of the present invention, the solution comprising ACMs formed in step b) of the process, may comprise magnesium, at a concentration selected from the group consisting of; 2 mmol/kg, 2.5 mmol/kg, 3 mmol/kg, 3.5 mmol/kg, 4 mmol/kg, 4.5 mmol/kg, 5 mmol/kg, 5.5 mmol/kg, 6 mmol/kg, 6.5 mmol/kg, 7 mmol/kg, 7.5 mmol/kg, and 8 mmol/kg.

In accordance with some embodiments of the process of the present invention, the solution comprising ACMs formed in step b) of the process, may comprise inorganic phosphate, at a concentration selected from the group consisting of; 10 mmol/kg, 11 mmol/kg, 12 mmol/kg, 13 mmol/kg, 14 mmol/kg, 15 mmol/kg, 16 mmol/kg, 17 mmol/kg, 18 mmol/kg, 19 mmol/kg, 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, and 30 mmol/kg.

In accordance with some embodiments of the process of the present invention, the solution comprising ACMs formed in step b) of the process, may comprise total phosphorus, at a concentration selected from the group consisting of; 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, 30 mmol/kg, 31 mmol/kg, 32 mmol/kg, 33 mmol/kg, 34 mmol/kg, 35 mmol/kg, 36 mmol/kg, 37 mmol/kg, 38 mmol/kg, 39 mmol/kg, and 40 mmol/kg.

In accordance with some embodiments of the process of the present invention, the solution comprising ACMs formed in step b) of the process, may comprise citrate, at a concentration selected from the group consisting of; 2 mmol/kg, 3 mmol/kg, 4 mmol/kg, 5 mmol/kg, 6 mmol/kg, 7 mmol/kg, 8 mmol/kg, 9 mmol/kg, 10 mmol/kg, 11 mmol/kg, 12 mmol/kg, 13 mmol/kg, 14 mmol/kg, 15 mmol/kg, 16 mmol/kg, 17 mmol/kg, 18 mmol/kg, 19 mmol/kg, and 20 mmol/kg.

In accordance with some embodiments of the process of the present invention, the solution comprising ACMs formed in step b) of the process, may comprise sodium, at a concentration selected from the group consisting of; 5 mmol/kg, 6 mmol/kg, 7 mmol/kg, 8 mmol/kg, 9 mmol/kg, 10 mmol/kg, 11 mmol/kg, 12 mmol/kg, 13 mmol/kg, 14 mmol/kg, 15 mmol/kg, 16 mmol/kg, 17 mmol/kg, 18 mmol/kg, 19 mmol/kg, 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, 30 mmol/kg, 31 mmol/kg, 32 mmol/kg, 33 mmol/kg, 34 mmol/kg, 35 mmol/kg, 36 mmol/kg, 37 mmol/kg, 38 mmol/kg, 39 mmol/kg, 40 mmol/kg, 41 mmol/kg, 42 mmol/kg, 43 mmol/kg, 44 mmol/kg, 45 mmol/kg, 46 mmol/kg, 47 mmol/kg, 48 mmol/kg, 49 mmol/kg, 50 mmol/kg, 51 mmol/kg, 52 mmol/kg, 53 mmol/kg, 54 mmol/kg, 55 mmol/kg, 56 mmol/kg, 57 mmol/kg, 58 mmol/kg, 59 mmol/kg, 60 mmol/kg, 61 mmol/kg, 62 mmol/kg, 63 mmol/kg, 64 mmol/kg, 65 mmol/kg, 66 mmol/kg, 67 mmol/kg, 68 mmol/kg, 69 mmol/kg, 70 mmol/kg, 71 mmol/kg, 72 mmol/kg, 73 mmol/kg, 74 mmol/kg, 75 mmol/kg, 76 mmol/kg, 77 mmol/kg, 78 mmol/kg, 79 mmol/kg, 80 mmol/kg, 81 mmol/kg, 82 mmol/kg, 83 mmol/kg, 84 mmol/kg, 85 mmol/kg, 86 mmol/kg, 87 mmol/kg, 88 mmol/kg, 89 mmol/kg, 90 mmol/kg, 91 mmol/kg, 92 mmol/kg, 93 mmol/kg, 94 mmol/kg, 95 mmol/kg, 96 mmol/kg, 97 mmol/kg, 98 mmol/kg, 99 mmol/kg, and 100 mmol/kg.

In accordance with some embodiments of the process of the present invention, the solution comprising ACMs formed in step b) of the process, may comprise potassium, at a concentration selected from the group consisting of; 10 mmol/kg, 11 mmol/kg, 12 mmol/kg, 13 mmol/kg, 14 mmol/kg, 15 mmol/kg, 16 mmol/kg, 17 mmol/kg, 18 mmol/kg, 19 mmol/kg, 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, 30 mmol/kg, 31 mmol/kg, 32 mmol/kg, 33 mmol/kg, 34 mmol/kg, 35 mmol/kg, 36 mmol/kg, 37 mmol/kg, 38 mmol/kg, 39 mmol/kg, 40 mmol/kg, 41 mmol/kg, 42 mmol/kg, 43 mmol/kg, 44 mmol/kg, 45 mmol/kg, 46 mmol/kg, 47 mmol/kg, 48 mmol/kg, 49 mmol/kg, and 50 mmol/kg.

In accordance with some embodiments of the process of the present invention, the solution comprising ACMs formed in step b) of the process, may comprise chloride, at a concentration selected from the group consisting of; 5 mmol/kg, 6 mmol/kg, 7 mmol/kg, 8 mmol/kg, 9 mmol/kg, 10 mmol/kg, 11 mmol/kg, 12 mmol/kg, 13 mmol/kg, 14 mmol/kg, 15 mmol/kg, 16 mmol/kg, 17 mmol/kg, 18 mmol/kg, 19 mmol/kg, 20 mmol/kg, 21 mmol/kg, 22 mmol/kg, 23 mmol/kg, 24 mmol/kg, 25 mmol/kg, 26 mmol/kg, 27 mmol/kg, 28 mmol/kg, 29 mmol/kg, 30 mmol/kg, 31 mmol/kg, 32 mmol/kg, 33 mmol/kg, 34 mmol/kg, 35 mmol/kg, 36 mmol/kg, 37 mmol/kg, 38 mmol/kg, 39 mmol/kg, 40 mmol/kg, 41 mmol/kg, 42 mmol/kg, 43 mmol/kg, 44 mmol/kg, 45 mmol/kg, 46 mmol/kg, 47 mmol/kg, 48 mmol/kg, 49 mmol/kg, 50 mmol/kg, 51 mmol/kg, 52 mmol/kg, 53 mmol/kg, 54 mmol/kg, 55 mmol/kg, 56 mmol/kg, 57 mmol/kg, 58 mmol/kg, 59 mmol/kg, 60 mmol/kg, 61 mmol/kg, 62 mmol/kg, 63 mmol/kg, 64 mmol/kg, 65 mmol/kg, 66 mmol/kg, 67 mmol/kg, 68 mmol/kg, 69 mmol/kg, 70 mmol/kg, 71 mmol/kg, 72 mmol/kg, 73 mmol/kg, 74 mmol/kg, 75 mmol/kg, 76 mmol/kg, 77 mmol/kg, 78 mmol/kg, 79 mmol/kg, 80 mmol/kg, 81 mmol/kg, 82 mmol/kg, 83 mmol/kg, 84 mmol/kg, 85 mmol/kg, 86 mmol/kg, 87 mmol/kg, 88 mmol/kg, 89 mmol/kg, 90 mmol/kg, 91 mmol/kg, 92 mmol/kg, 93 mmol/kg, 94 mmol/kg, 95 mmol/kg, 96 mmol/kg, 97 mmol/kg, 98 mmol/kg, 99 mmol/kg, and 100 mmol/kg.

In accordance with some embodiments of the process of the present invention, the solution comprising ACMs formed in step b) of the process, may comprise total caseins at a concentration selected from the group consisting of; 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, and 100 g/L.

In some embodiments, the Z-average diameter of the artificial casein micelles is greater than 30 nm. The Z-average diameter of the artificial casein micelles of the present invention may be determined, for example and without limitation, via dynamic light scattering measurements or via scanning electron microscopy (SEM) as performed in the examples herein. Without limitation, the Z-average diameter of the artificial casein micelles of the present invention may be greater than; 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, or 300 nm.

In preferred embodiments, the Z-average diameter of the artificial casein micelles falls within the range of 40 to 500 nm. Without limitation, the solution comprising ACMs formed in step b) comprises ACMs with a Z-average diameter falling within any range selected from the group of ranges consisting of; 40 to 500 nm, 45 to 500 nm, 50 to 500 nm, 55 to 500 nm, 60 to 500 nm, 65 to 500 nm, 70 to 500 nm, 75 to 500 nm, 80 to 500 nm, 85 to 500 nm, 90 to 500 nm, 95 to 500 nm, 100 to 500 nm, 105 to 500 nm, 110 to 500 nm, 115 to 500 nm, 120 to 500 nm, 125 to 500 nm, 130 to 500 nm, 135 to 500 nm, 140 to 500 nm, 145 to 500 nm, 150 to 500 nm, 155 to 500 nm, 160 to 500 nm, 165 to 500 nm, 170 to 500 nm, 175 to 500 nm, 180 to 500 nm, 185 to 500 nm, 190 to 500 nm, 195 to 500 nm, 200 to 500 nm, 205 to 500 nm, 210 to 500 nm, 215 to 500 nm, 220 to 500 nm, 225 to 500 nm, 230 to 500 nm, 235 to 500 nm, 240 to 500 nm, 245 to 500 nm, 250 to 500 nm, 255 to 500 nm, 260 to 500 nm, 265 to 500 nm, 270 to 500 nm, 275 to 500 nm, 280 to 500 nm, 285 to 500 nm, 290 to 500 nm, 295 to 500 nm, 300 to 500 nm, 305 to 500 nm, 310 to 500 nm, 315 to 500 nm, 320 to 500 nm, 325 to 500 nm, 330 to 500 nm, 335 to 500 nm, 340 to 500 nm, 345 to 500 nm, 350 to 500 nm, 355 to 500 nm, 360 to 500 nm, 365 to 500 nm, 370 to 500 nm, 375 to 500 nm, 380 to 500 nm, 385 to 500 nm, 390 to 500 nm, 395 to 500 nm, 400 to 500 nm, 405 to 500 nm, 410 to 500 nm, 415 to 500 nm, 420 to 500 nm, 425 to 500 nm, 430 to 500 nm, 435 to 500 nm, 440 to 500 nm, 445 to 500 nm, 450 to 500 nm, 455 to 500 nm, 460 to 500 nm, 465 to 500 nm, 470 to 500 nm, 475 to 500 nm, 480 to 500 nm, 485 to 500 nm, 490 to 500 nm, and 495 to 500 nm.

Preferably, the solution comprising ACMs formed in step b) comprises ACMs with a Z-average diameter selected from the group consisting of; 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, and 500 nm.

In some embodiments, the solution comprising ACMs formed in step b) comprises ACMs with hydration values (g water/g micellar casein) selected from the group consisting of; 1 (g water/g micellar casein), 1.1 (g water/g micellar casein), 1.2 (g water/g micellar casein), 1.3 (g water/g micellar casein), 1.4 (g water/g micellar casein), 1.5 (g water/g micellar casein), 1.6 (g water/g micellar casein), 1.7 (g water/g micellar casein), 1.8 (g water/g micellar casein), 1.9 (g water/g micellar casein), 2 (g water/g micellar casein), 2.1 (g water/g micellar casein), 2.2 (g water/g micellar casein), 2.3 (g water/g micellar casein), 2.4 (g water/g micellar casein), 2.5 (g water/g micellar casein), 2.6 (g water/g micellar casein), 2.7 (g water/g micellar casein), 2.8 (g water/g micellar casein), 2.9 (g water/g micellar casein), 3 (g water/g micellar casein), 3.1 (g water/g micellar casein), 3.2 (g water/g micellar casein), 3.3 (g water/g micellar casein), 3.4 (g water/g micellar casein), 3.5 (g water/g micellar casein), 3.6 (g water/g micellar casein), 3.7 (g water/g micellar casein), 3.8 (g water/g micellar casein), 3.9 (g water/g micellar casein), 4 (g water/g micellar casein), 4.1 (g water/g micellar casein), 4.2 (g water/g micellar casein), 4.3 (g water/g micellar casein), 4.4 (g water/g micellar casein), 4.5 (g water/g micellar casein), 4.6 (g water/g micellar casein), 4.7 (g water/g micellar casein), 4.8 (g water/g micellar casein), 4.9 (g water/g micellar casein), 5 (g water/g micellar casein), 5.1 (g water/g micellar casein), 5.2 (g water/g micellar casein), 5.3 (g water/g micellar casein), 5.4 (g water/g micellar casein), 5.5 (g water/g micellar casein), 5.6 (g water/g micellar casein), 5.7 (g water/g micellar casein), 5.8 (g water/g micellar casein), 5.9 (g water/g micellar casein), 6 (g water/g micellar casein), 6.1 (g water/g micellar casein), 6.2 (g water/g micellar casein), 6.3 (g water/g micellar casein), 6.4 (g water/g micellar casein), 6.5 (g water/g micellar casein), 6.6 (g water/g micellar casein), 6.7 (g water/g micellar casein), 6.8 (g water/g micellar casein), 6.9 (g water/g micellar casein), 7 (g water/g micellar casein), 7.1 (g water/g micellar casein), 7.2 (g water/g micellar casein), 7.3 (g water/g micellar casein), 7.4 (g water/g micellar casein), 7.5 (g water/g micellar casein), 7.6 (g water/g micellar casein), 7.7 (g water/g micellar casein), 7.8 (g water/g micellar casein), 7.9 (g water/g micellar casein), and 8 (g water/g micellar casein).

In a preferred embodiment, the process of the present invention is conducted on an industrial scale; and/or is conducted continuously; and/or is conducted on a scale capable of producing an amount of the solution comprising ACMs formed in step b) in a single batch, or process run, wherein the capable amount of the solution comprising ACMs formed in step b) in a single batch, or process run is selected from the group consisting of; 100 L, 110 L, 120 L, 130 L, 140 L, 150 L, 160 L, 170 L, 180 L, 190 L, 200 L, 210 L, 220 L, 230 L, 240 L, 250 L, 260 L, 270 L, 280 L, 290 L, 300 L, 310 L, 320 L, 330 L, 340 L, 350 L, 360 L, 370 L, 380 L, 390 L, 400 L, 410 L, 420 L, 430 L, 440 L, 450 L, 460 L, 470 L, 480 L, 490 L, 500 L, 510 L, 520 L, 530 L, 540 L, 550 L, 560 L, 570 L, 580 L, 590 L, 600 L, 610 L, 620 L, 630 L, 640 L, 650 L, 660 L, 670 L, 680 L, 690 L, 700 L, 710 L, 720 L, 730 L, 740 L, 750 L, 760 L, 770 L, 780 L, 790 L, 800 L, 810 L, 820 L, 830 L, 840 L, 850 L, 860 L, 870 L, 880 L, 890 L, 900 L, 910 L, 920 L, 930 L, 940 L, 950 L, 960 L, 970 L, 980 L, 990 L, 1000 L, 1010 L, 1020 L, 1030 L, 1040 L, 1050 L, 1060 L, 1070 L, 1080 L, 1090 L, 1100 L, 1110 L, 1120 L, 1130 L, 1140 L, 1150 L, 1160 L, 1170 L, 1180 L, 1190 L, 1200 L, 1210 L, 1220 L, 1230 L, 1240 L, 1250 L, 1260 L, 1270 L, 1280 L, 1290 L, 1300 L, 1310 L, 1320 L, 1330 L, 1340 L, 1350 L, 1360 L, 1370 L, 1380 L, 1390 L, 1400 L, 1410 L, 1420 L, 1430 L, 1440 L, 1450 L, 1460 L, 1470 L, 1480 L, 1490 L, 1500 L, 1510 L, 1520 L, 1530 L, 1540 L, 1550 L, 1560 L, 1570 L, 1580 L, 1590 L, 1600 L, 1610 L, 1620 L, 1630 L, 1640 L, 1650 L, 1660 L, 1670 L, 1680 L, 1690 L, 1700 L, 1710 L, 1720 L, 1730 L, 1740 L, 1750 L, 1760 L, 1770 L, 1780 L, 1790 L, 1800 L, 1810 L, 1820 L, 1830 L, 1840 L, 1850 L, 1860 L, 1870 L, 1880 L, 1890 L, 1900 L, 1910 L, 1920 L, 1930 L, 1940 L, 1950 L, 1960 L, 1970 L, 1980 L, 1990 L, 2000 L, 2010 L, 2020 L, 2030 L, 2040 L, 2050 L, 2060 L, 2070 L, 2080 L, 2090 L, 2100 L, 2110 L, 2120 L, 2130 L, 2140 L, 2150 L, 2160 L, 2170 L, 2180 L, 2190 L, 2200 L, 2210 L, 2220 L, 2230 L, 2240 L, 2250 L, 2260 L, 2270 L, 2280 L, 2290 L, 2300 L, 2310 L, 2320 L, 2330 L, 2340 L, 2350 L, 2360 L, 2370 L, 2380 L, 2390 L, 2400 L, 2410 L, 2420 L, 2430 L, 2440 L, 2450 L, 2460 L, 2470 L, 2480 L, 2490 L, 2500 L, 2510 L, 2520 L, 2530 L, 2540 L, 2550 L, 2560 L, 2570 L, 2580 L, 2590 L, 2600 L, 2610 L, 2620 L, 2630 L, 2640 L, 2650 L, 2660 L, 2670 L, 2680 L, 2690 L, 2700 L, 2710 L, 2720 L, 2730 L, 2740 L, 2750 L, 2760 L, 2770 L, 2780 L, 2790 L, 2800 L, 2810 L, 2820 L, 2830 L, 2840 L, 2850 L, 2860 L, 2870 L, 2880 L, 2890 L, 2900 L, 2910 L, 2920 L, 2930 L, 2940 L, 2950 L, 2960 L, 2970 L, 2980 L, 2990 L, 3000 L, 3010 L, 3020 L, 3030 L, 3040 L, 3050 L, 3060 L, 3070 L, 3080 L, 3090 L, 3100 L, 3110 L, 3120 L, 3130 L, 3140 L, 3150 L, 3160 L, 3170 L, 3180 L, 3190 L, 3200 L, 3210 L, 3220 L, 3230 L, 3240 L, 3250 L, 3260 L, 3270 L, 3280 L, 3290 L, 3300 L, 3310 L, 3320 L, 3330 L, 3340 L, 3350 L, 3360 L, 3370 L, 3380 L, 3390 L, 3400 L, 3410 L, 3420 L, 3430 L, 3440 L, 3450 L, 3460 L, 3470 L, 3480 L, 3490 L, 3500 L, 3510 L, 3520 L, 3530 L, 3540 L, 3550 L, 3560 L, 3570 L, 3580 L, 3590 L, 3600 L, 3610 L, 3620 L, 3630 L, 3640 L, 3650 L, 3660 L, 3670 L, 3680 L, 3690 L, 3700 L, 3710 L, 3720 L, 3730 L, 3740 L, 3750 L, 3760 L, 3770 L, 3780 L, 3790 L, 3800 L, 3810 L, 3820 L, 3830 L, 3840 L, 3850 L, 3860 L, 3870 L, 3880 L, 3890 L, 3900 L, 3910 L, 3920 L, 3930 L, 3940 L, 3950 L, 3960 L, 3970 L, 3980 L, 3990 L, 4000 L, 4010 L, 4020 L, 4030 L, 4040 L, 4050 L, 4060 L, 4070 L, 4080 L, 4090 L, 4100 L, 4110 L, 4120 L, 4130 L, 4140 L, 4150 L, 4160 L, 4170 L, 4180 L, 4190 L, 4200 L, 4210 L, 4220 L, 4230 L, 4240 L, 4250 L, 4260 L, 4270 L, 4280 L, 4290 L, 4300 L, 4310 L, 4320 L, 4330 L, 4340 L, 4350 L, 4360 L, 4370 L, 4380 L, 4390 L, 4400 L, 4410 L, 4420 L, 4430 L, 4440 L, 4450 L, 4460 L, 4470 L, 4480 L, 4490 L, 4500 L, 4510 L, 4520 L, 4530 L, 4540 L, 4550 L, 4560 L, 4570 L, 4580 L, 4590 L, 4600 L, 4610 L, 4620 L, 4630 L, 4640 L, 4650 L, 4660 L, 4670 L, 4680 L, 4690 L, 4700 L, 4710 L, 4720 L, 4730 L, 4740 L, 4750 L, 4760 L, 4770 L, 4780 L, 4790 L, 4800 L, 4810 L, 4820 L, 4830 L, 4840 L, 4850 L, 4860 L, 4870 L, 4880 L, 4890 L, 4900 L, 4910 L, 4920 L, 4930 L, 4940 L, 4950 L, 4960 L, 4970 L, 4980 L, 4990 L, 5000 L, 5010 L, 5020 L, 5030 L, 5040 L, 5050 L, 5060 L, 5070 L, 5080 L, 5090 L, 5100 L, 5110 L, 5120 L, 5130 L, 5140 L, 5150 L, 5160 L, 5170 L, 5180 L, 5190 L, 5200 L, 5210 L, 5220 L, 5230 L, 5240 L, 5250 L, 5260 L, 5270 L, 5280 L, 5290 L, 5300 L, 5310 L, 5320 L, 5330 L, 5340 L, 5350 L, 5360 L, 5370 L, 5380 L, 5390 L, 5400 L, 5410 L, 5420 L, 5430 L, 5440 L, 5450 L, 5460 L, 5470 L, 5480 L, 5490 L, 5500 L, 5510 L, 5520 L, 5530 L, 5540 L, 5550 L, 5560 L, 5570 L, 5580 L, 5590 L, 5600 L, 5610 L, 5620 L, 5630 L, 5640 L, 5650 L, 5660 L, 5670 L, 5680 L, 5690 L, 5700 L, 5710 L, 5720 L, 5730 L, 5740 L, 5750 L, 5760 L, 5770 L, 5780 L, 5790 L, 5800 L, 5810 L, 5820 L, 5830 L, 5840 L, 5850 L, 5860 L, 5870 L, 5880 L, 5890 L, 5900 L, 5910 L, 5920 L, 5930 L, 5940 L, 5950 L, 5960 L, 5970 L, 5980 L, 5990 L, 6000 L, 6010 L, 6020 L, 6030 L, 6040 L, 6050 L, 6060 L, 6070 L, 6080 L, 6090 L, 6100 L, 6110 L, 6120 L, 6130 L, 6140 L, 6150 L, 6160 L, 6170 L, 6180 L, 6190 L, 6200 L, 6210 L, 6220 L, 6230 L, 6240 L, 6250 L, 6260 L, 6270 L, 6280 L, 6290 L, 6300 L, 6310 L, 6320 L, 6330 L, 6340 L, 6350 L, 6360 L, 6370 L, 6380 L, 6390 L, 6400 L, 6410 L, 6420 L, 6430 L, 6440 L, 6450 L, 6460 L, 6470 L, 6480 L, 6490 L, 6500 L, 6510 L, 6520 L, 6530 L, 6540 L, 6550 L, 6560 L, 6570 L, 6580 L, 6590 L, 6600 L, 6610 L, 6620 L, 6630 L, 6640 L, 6650 L, 6660 L, 6670 L, 6680 L, 6690 L, 6700 L, 6710 L, 6720 L, 6730 L, 6740 L, 6750 L, 6760 L, 6770 L, 6780 L, 6790 L, 6800 L, 6810 L, 6820 L, 6830 L, 6840 L, 6850 L, 6860 L, 6870 L, 6880 L, 6890 L, 6900 L, 6910 L, 6920 L, 6930 L, 6940 L, 6950 L, 6960 L, 6970 L, 6980 L, 6990 L, 7000 L, 7010 L, 7020 L, 7030 L, 7040 L, 7050 L, 7060 L, 7070 L, 7080 L, 7090 L, 7100 L, 7110 L, 7120 L, 7130 L, 7140 L, 7150 L, 7160 L, 7170 L, 7180 L, 7190 L, 7200 L, 7210 L, 7220 L, 7230 L, 7240 L, 7250 L, 7260 L, 7270 L, 7280 L, 7290 L, 7300 L, 7310 L, 7320 L, 7330 L, 7340 L, 7350 L, 7360 L, 7370 L, 7380 L, 7390 L, 7400 L, 7410 L, 7420 L, 7430 L, 7440 L, 7450 L, 7460 L, 7470 L, 7480 L, 7490 L, 7500 L, 7510 L, 7520 L, 7530 L, 7540 L, 7550 L, 7560 L, 7570 L, 7580 L, 7590 L, 7600 L, 7610 L, 7620 L, 7630 L, 7640 L, 7650 L, 7660 L, 7670 L, 7680 L, 7690 L, 7700 L, 7710 L, 7720 L, 7730 L, 7740 L, 7750 L, 7760 L, 7770 L, 7780 L, 7790 L, 7800 L, 7810 L, 7820 L, 7830 L, 7840 L, 7850 L, 7860 L, 7870 L, 7880 L, 7890 L, 7900 L, 7910 L, 7920 L, 7930 L, 7940 L, 7950 L, 7960 L, 7970 L, 7980 L, 7990 L, 8000 L, 8010 L, 8020 L, 8030 L, 8040 L, 8050 L, 8060 L, 8070 L, 8080 L, 8090 L, 8100 L, 8110 L, 8120 L, 8130 L, 8140 L, 8150 L, 8160 L, 8170 L, 8180 L, 8190 L, 8200 L, 8210 L, 8220 L, 8230 L, 8240 L, 8250 L, 8260 L, 8270 L, 8280 L, 8290 L, 8300 L, 8310 L, 8320 L, 8330 L, 8340 L, 8350 L, 8360 L, 8370 L, 8380 L, 8390 L, 8400 L, 8410 L, 8420 L, 8430 L, 8440 L, 8450 L, 8460 L, 8470 L, 8480 L, 8490 L, 8500 L, 8510 L, 8520 L, 8530 L, 8540 L, 8550 L, 8560 L, 8570 L, 8580 L, 8590 L, 8600 L, 8610 L, 8620 L, 8630 L, 8640 L, 8650 L, 8660 L, 8670 L, 8680 L, 8690 L, 8700 L, 8710 L, 8720 L, 8730 L, 8740 L, 8750 L, 8760 L, 8770 L, 8780 L, 8790 L, 8800 L, 8810 L, 8820 L, 8830 L, 8840 L, 8850 L, 8860 L, 8870 L, 8880 L, 8890 L, 8900 L, 8910 L, 8920 L, 8930 L, 8940 L, 8950 L, 8960 L, 8970 L, 8980 L, 8990 L, 9000 L, 9010 L, 9020 L, 9030 L, 9040 L, 9050 L, 9060 L, 9070 L, 9080 L, 9090 L, 9100 L, 9110 L, 9120 L, 9130 L, 9140 L, 9150 L, 9160 L, 9170 L, 9180 L, 9190 L, 9200 L, 9210 L, 9220 L, 9230 L, 9240 L, 9250 L, 9260 L, 9270 L, 9280 L, 9290 L, 9300 L, 9310 L, 9320 L, 9330 L, 9340 L, 9350 L, 9360 L, 9370 L, 9380 L, 9390 L, 9400 L, 9410 L, 9420 L, 9430 L, 9440 L, 9450 L, 9460 L, 9470 L, 9480 L, 9490 L, 9500 L, 9510 L, 9520 L, 9530 L, 9540 L, 9550 L, 9560 L, 9570 L, 9580 L, 9590 L, 9600 L, 9610 L, 9620 L, 9630 L, 9640 L, 9650 L, 9660 L, 9670 L, 9680 L, 9690 L, 9700 L, 9710 L, 9720 L, 9730 L, 9740 L, 9750 L, 9760 L, 9770 L, 9780 L, 9790 L, 9800 L, 9810 L, 9820 L, 9830 L, 9840 L, 9850 L, 9860 L, 9870 L, 9880 L, 9890 L, 9900 L, 9910 L, 9920 L, 9930 L, 9940 L, 9950 L, 9960 L, 9970 L, 9980 L, 9990 L, and 10000 L.

In one embodiment, the disclosure herein provides a curds composition comprising the micellar solution of the present invention, in coagulated form.

The curds composition produced by the process of the present invention is a useful precursor for the manufacture of downstream products such as yogurt or cheese. The curds composition may be coagulated by the action of an acid or a renneting agent or a milk-clotting enzyme, such as, but not limited to, aspartic protease, serine protease or cysteine protease. Suitable acids for coagulation include, without limitation, citric acid vinegar, and lactic acid.

In some embodiments, a yogurt composition may be formed using the methods described herein. The yogurt may be formed using the micellar solution described herein. The method may comprise heating and then cooling the micellar solution and acidifying the micellar solution with an acid or a microorganism. The microorganism may comprise one or more of Lactobacillus delbrueckii subsp. bulgaricus, Streptococcus thermophilus, a lactobacilli or a bifidobacteria.

In some embodiments, following acidification, a renneting agent may be added to form a renneted curd (coagulated curd matrix), which may then be used to make cheese. Micelles in a micellar solution, such as milk and also the ACM solutions described and produced herein, are stable and repel each other in colloidal suspension. In presence of renneting agents or milk-clotting enzymes, and when acidified, micelles are destabilized and thus coagulate. In presence of renneting agents or milk-clotting enzymes, a cross-linked coagulated curd matrix is formed.

In some embodiments, the curds composition further comprises a renneting agent. Renneting agents suitable for performance of the present invention include, without limitation, protease enzymes, chymosin, pepsin, lipase, animal derived rennet, plant derived rennet (including extracts from Galium spp., dried caper leaves, nettles, thistles, mallow, Withania coagulans, ground ivy, Cynara, soy), calf rennet, kid goat rennet, fungi derived rennet, microbially derived rennet (eg; extracts of Rhizomucor miehei) and recombinantly produced chymosin.

In a preferred embodiment, the curds composition has a Maximum G′ (storage modulus) falling within the range of 50 to 400 Pa, preferably after 1 hour incubation with rennet. Without limitation, the Maximum G′ of the curds composition of the present invention may be achieved after any period of incubation with rennet, selected from the group consisting of; 0.1 hr, 0.2 hr, 0.3 hr, 0.4 hr, 0.5 hr, 0.6 hr, 0.7 hr, 0.8 hr, 0.9 hr, 1 hr, 1.1 hr, 1.2 hr, 1.3 hr, 1.4 hr, 1.5 hr, 1.6 hr, 1.7 hr, 1.8 hr, 1.9 hr, 2 hr, 2.1 hr, 2.2 hr, 2.3 hr, 2.4 hr, 2.5 hr, 2.6 hr, 2.7 hr, 2.8 hr, 2.9 hr, 3 hr, 3.1 hr, 3.2 hr, 3.3 hr, 3.4 hr, 3.5 hr, 3.6 hr, 3.7 hr, 3.8 hr, 3.9 hr, 4 hr, 4.1 hr, 4.2 hr, 4.3 hr, 4.4 hr, 4.5 hr, 4.6 hr, 4.7 hr, 4.8 hr, 4.9 hr, 5 hr, 5.1 hr, 5.2 hr, 5.3 hr, 5.4 hr, 5.5 hr, 5.6 hr, 5.7 hr, 5.8 hr, 5.9 hr, and 6 hr.

In a preferred embodiment, the curds composition has a Maximum G′ (storage modulus) selected from the group of ranges consisting of; 50 to 400 Pa, 55 to 400 Pa, 60 to 400 Pa, 65 to 400 Pa, 70 to 400 Pa, 75 to 400 Pa, 80 to 400 Pa, 85 to 400 Pa, 90 to 400 Pa, 95 to 400 Pa, 100 to 400 Pa, 105 to 400 Pa, 110 to 400 Pa, 115 to 400 Pa, 120 to 400 Pa, 125 to 400 Pa, 130 to 400 Pa, 135 to 400 Pa, 140 to 400 Pa, 145 to 400 Pa, 150 to 400 Pa, 155 to 400 Pa, 160 to 400 Pa, 165 to 400 Pa, 170 to 400 Pa, 175 to 400 Pa, 180 to 400 Pa, 185 to 400 Pa, 190 to 400 Pa, 195 to 400 Pa, 200 to 400 Pa, 205 to 400 Pa, 210 to 400 Pa, 215 to 400 Pa, 220 to 400 Pa, 225 to 400 Pa, 230 to 400 Pa, 235 to 400 Pa, 240 to 400 Pa, 245 to 400 Pa, 250 to 400 Pa, 255 to 400 Pa, 260 to 400 Pa, 265 to 400 Pa, 270 to 400 Pa, 275 to 400 Pa, 280 to 400 Pa, 285 to 400 Pa, 290 to 400 Pa, 295 to 400 Pa, 300 to 400 Pa, 305 to 400 Pa, 310 to 400 Pa, 315 to 400 Pa, 320 to 400 Pa, 325 to 400 Pa, 330 to 400 Pa, 335 to 400 Pa, 340 to 400 Pa, 345 to 400 Pa, 350 to 400 Pa, 355 to 400 Pa, 360 to 400 Pa, 365 to 400 Pa, 370 to 400 Pa, 375 to 400 Pa, 380 to 400 Pa, 385 to 400 Pa, 390 to 400 Pa, and 395 to 400 Pa.

In a most preferred embodiment, the curds composition has a Maximum G′ (storage modulus) selected from the group consisting of; 50 Pa, 51 Pa, 52 Pa, 53 Pa, 54 Pa, 55 Pa, 56 Pa, 57 Pa, 58 Pa, 59 Pa, 60 Pa, 61 Pa, 62 Pa, 63 Pa, 64 Pa, 65 Pa, 66 Pa, 67 Pa, 68 Pa, 69 Pa, 70 Pa, 71 Pa, 72 Pa, 73 Pa, 74 Pa, 75 Pa, 76 Pa, 77 Pa, 78 Pa, 79 Pa, 80 Pa, 81 Pa, 82 Pa, 83 Pa, 84 Pa, 85 Pa, 86 Pa, 87 Pa, 88 Pa, 89 Pa, 90 Pa, 91 Pa, 92 Pa, 93 Pa, 94 Pa, 95 Pa, 96 Pa, 97 Pa, 98 Pa, 99 Pa, 100 Pa, 101 Pa, 102 Pa, 103 Pa, 104 Pa, 105 Pa, 106 Pa, 107 Pa, 108 Pa, 109 Pa, 110 Pa, 111 Pa, 112 Pa, 113 Pa, 114 Pa, 115 Pa, 116 Pa, 117 Pa, 118 Pa, 119 Pa, 120 Pa, 121 Pa, 122 Pa, 123 Pa, 124 Pa, 125 Pa, 126 Pa, 127 Pa, 128 Pa, 129 Pa, 130 Pa, 131 Pa, 132 Pa, 133 Pa, 134 Pa, 135 Pa, 136 Pa, 137 Pa, 138 Pa, 139 Pa, 140 Pa, 141 Pa, 142 Pa, 143 Pa, 144 Pa, 145 Pa, 146 Pa, 147 Pa, 148 Pa, 149 Pa, 150 Pa, 151 Pa, 152 Pa, 153 Pa, 154 Pa, 155 Pa, 156 Pa, 157 Pa, 158 Pa, 159 Pa, 160 Pa, 161 Pa, 162 Pa, 163 Pa, 164 Pa, 165 Pa, 166 Pa, 167 Pa, 168 Pa, 169 Pa, 170 Pa, 171 Pa, 172 Pa, 173 Pa, 174 Pa, 175 Pa, 176 Pa, 177 Pa, 178 Pa, 179 Pa, 180 Pa, 181 Pa, 182 Pa, 183 Pa, 184 Pa, 185 Pa, 186 Pa, 187 Pa, 188 Pa, 189 Pa, 190 Pa, 191 Pa, 192 Pa, 193 Pa, 194 Pa, 195 Pa, 196 Pa, 197 Pa, 198 Pa, 199 Pa, 200 Pa, 201 Pa, 202 Pa, 203 Pa, 204 Pa, 205 Pa, 206 Pa, 207 Pa, 208 Pa, 209 Pa, 210 Pa, 211 Pa, 212 Pa, 213 Pa, 214 Pa, 215 Pa, 216 Pa, 217 Pa, 218 Pa, 219 Pa, 220 Pa, 221 Pa, 222 Pa, 223 Pa, 224 Pa, 225 Pa, 226 Pa, 227 Pa, 228 Pa, 229 Pa, 230 Pa, 231 Pa, 232 Pa, 233 Pa, 234 Pa, 235 Pa, 236 Pa, 237 Pa, 238 Pa, 239 Pa, 240 Pa, 241 Pa, 242 Pa, 243 Pa, 244 Pa, 245 Pa, 246 Pa, 247 Pa, 248 Pa, 249 Pa, 250 Pa, 251 Pa, 252 Pa, 253 Pa, 254 Pa, 255 Pa, 256 Pa, 257 Pa, 258 Pa, 259 Pa, 260 Pa, 261 Pa, 262 Pa, 263 Pa, 264 Pa, 265 Pa, 266 Pa, 267 Pa, 268 Pa, 269 Pa, 270 Pa, 271 Pa, 272 Pa, 273 Pa, 274 Pa, 275 Pa, 276 Pa, 277 Pa, 278 Pa, 279 Pa, 280 Pa, 281 Pa, 282 Pa, 283 Pa, 284 Pa, 285 Pa, 286 Pa, 287 Pa, 288 Pa, 289 Pa, 290 Pa, 291 Pa, 292 Pa, 293 Pa, 294 Pa, 295 Pa, 296 Pa, 297 Pa, 298 Pa, 299 Pa, 300 Pa, 301 Pa, 302 Pa, 303 Pa, 304 Pa, 305 Pa, 306 Pa, 307 Pa, 308 Pa, 309 Pa, 310 Pa, 311 Pa, 312 Pa, 313 Pa, 314 Pa, 315 Pa, 316 Pa, 317 Pa, 318 Pa, 319 Pa, 320 Pa, 321 Pa, 322 Pa, 323 Pa, 324 Pa, 325 Pa, 326 Pa, 327 Pa, 328 Pa, 329 Pa, 330 Pa, 331 Pa, 332 Pa, 333 Pa, 334 Pa, 335 Pa, 336 Pa, 337 Pa, 338 Pa, 339 Pa, 340 Pa, 341 Pa, 342 Pa, 343 Pa, 344 Pa, 345 Pa, 346 Pa, 347 Pa, 348 Pa, 349 Pa, 350 Pa, 351 Pa, 352 Pa, 353 Pa, 354 Pa, 355 Pa, 356 Pa, 357 Pa, 358 Pa, 359 Pa, 360 Pa, 361 Pa, 362 Pa, 363 Pa, 364 Pa, 365 Pa, 366 Pa, 367 Pa, 368 Pa, 369 Pa, 370 Pa, 371 Pa, 372 Pa, 373 Pa, 374 Pa, 375 Pa, 376 Pa, 377 Pa, 378 Pa, 379 Pa, 380 Pa, 381 Pa, 382 Pa, 383 Pa, 384 Pa, 385 Pa, 386 Pa, 387 Pa, 388 Pa, 389 Pa, 390 Pa, 391 Pa, 392 Pa, 393 Pa, 394 Pa, 395 Pa, 396 Pa, 397 Pa, 398 Pa, 399 Pa, and 400 Pa.

In a further embodiment, the disclosure herein provides an edible composition comprising the micellar solution of the present invention, or the curds composition of the present invention. Such edible compositions include, without limitation, yogurts, cheeses and milk substitutes.

In some embodiments, the edible composition does not contain any animal-derived protein.

In one embodiment, the disclosure herein provides a method for producing an edible composition, comprising; subjecting the ACM solution formed in step b) of the process of the present invention to a first condition to form coagulates.

In some embodiments of the method for producing an edible composition, the first condition is the addition of acid or acidification of the micellar solution with a microorganism.

In some embodiments of the method for producing an edible composition, the method further comprises subjecting the coagulates to a renneting agent to form a renneted curd.

In some embodiments of the method for producing an edible composition, the method further comprises aging and/or maturing the renneted curd to form a cheese composition. The renneted curd may be further treated to create a cheese or cheese like product. In some cases, such as a mozzarella product, the renneted curd may be heated and stretched. In other embodiments, the renneted curd is aged, such as for brie, camembert, feta, halloumi, gouda, edam, cheddar, manchego, swiss, colby, muenster, blue cheese or parmesan type cheese or cheese-like product.

In some embodiments, the micellar solution or renneted curd may be treated with hot water for the formation of cheese, such as for mozzarella-type cheese. Hot water treatment may be performed at a temperature of about 50° C. to about 90° C. Hot water treatment may be performed at a temperature of at least 55° C. Hot water treatment may be performed at a temperature of at most 75° C. Hot water treatment may be performed at a temperature of 50° C. to 55° C., 55° C. to 60° C., 55° C. to 65° C., 55° C. to 70° C., 55° C. to 75° C., 60° C. to 65° C., 60° C. to 70° C., 60° C. to 75° C., 65° C. to 70° C., 65° C. to 75° C., 70° C. to 75° C., 75° C. to 80° C., 80° C. to 85° C., or 85° C. to 90° C. Hot water treatment may be performed at a temperature of about 50° C., about 55° C., about 60° C., about 65° C., about 70° C., about 75° C., about 80° C., about 85° C. or about 90° C. Hot water treatment may be performed at a temperature of at least 50° C., 55° C., 60° C., 65° C., 70° C., 75° C., 80° C., or 85° C. Hot water treatment may be performed at a temperature of at most 55° C., 60° C., 65° C., 70° C., 75° C., 80° C., 85° C. or 90° C. In some cases, after hot water treatment, the product is stretched into a cheese.

In some embodiments of the method for producing an edible composition, the edible composition does not contain any animal-derived protein.

Cheese compositions formed using the methods described herein optionally may not comprise any animal-derived components for example, where recombinantly derived casein proteins are utilised. Cheese compositions formed using the methods described herein may optionally not comprise any animal-derived dairy-based components, such as animal-derived dairy proteins. Cheese compositions formed using the methods described herein may optionally not comprise any whey proteins. Cheese compositions formed using the methods described herein may optionally not comprise one or more casein proteins (α, β, or κ). Cheese compositions described herein may be pasta-filata like cheese such as mozzarella cheese. Soft cheeses such as paneer, cream cheese or cottage cheese may also be formed using the methods described herein. Other types of cheese such as aged and ripened cheeses may also be formed using the methods described herein, such as brie, camembert, feta, halloumi, gouda, edam, cheddar, manchego, swiss, colby, muenster, blue cheese and parmesan.

The texture of a cheese made by methods described herein may be comparable to the texture of a similar type of cheese made using animal-derived dairy derived proteins, such as cheese made from animal milk. Texture of a cheese may be tested using a trained panel of human subjects or machines such as a texture analyzer.

The taste of a cheese made by methods described herein may be comparable to a similar type of cheese made using animal-derived dairy proteins. Taste of a cheese may be tested using a trained panel of human subjects.

Cheese compositions described herein may have a browning ability which is comparable to a similar type of cheese made using animal-derived dairy proteins. Cheese compositions described herein may have a melting ability which is comparable to a similar type of cheese made using animal-derived dairy proteins.

The texture of a yogurt made by methods described herein may be comparable to the texture of a similar type of yogurt made using animal-derived dairy derived proteins, such as yogurt made from animal milk. Texture of a yogurt may be tested using a trained panel of human subjects or machines such as a texture analyzer.

The taste of a yogurt made by methods described herein may be comparable to a similar type of yogurt made using animal-derived dairy proteins. Taste of a yogurt may be tested using a trained panel of human subjects.

### EXAMPLES

### Materials

Bovine sodium caseinate (Lactonat EN, 90.2% protein) was provided by Lactoprot (Lactoprot Deutschland GmbH, Kaltenkirchen, Germany).

Citric acid (C0759), calcium chloride (C1016), magnesium chloride (M8266), potassium phosphate monobasic (P5379), potassium chloride (104936), potassium carbonate (104928), potassium sulfate (105153), trisodium citrate dihydrate (S4641), magnesium citrate tribasic nonahydrate (63067), guanidine hydrochloride (50950), sodium chloride (31434-M), sodium hydroxide (221465), lactic acid 85% (252476), sodium phosphate dibasic (S7907), L-dithiothreitol (D9760), hydrochloric acid fuming 37% (113386), nitric acid 65% (100456), hydrogen peroxide 30% (107209), silicone grease (107746), Sodium phosphate dibasic dihydrate (1.06580), citric acid monohydrate (1.00244), ethanol absolute (1.00983) and standards of calcium (1.70308), magnesium (1.70331), phosphorus (1.70340), sodium (1.70353) and potassium (1.70342) with a concentration of 1000 mg/L were bought from Sigma-Aldrich (Saint Louis, MO, USA).

Acetonitrile ULC-MS was obtained from Actu-All (Oss, The Netherlands). Osmium tetroxide (19134), 50% glutaraldehyde solution (16316-10), and carbon adhesive tabs (77825-12) were bought from EMS (Electron Microscopy Sciences, Hatfield, PA, USA).

Trifluoroacectic acid (76051) was purchased from Alfa Aesar (Ward Hill, MA, USA). Tripotassium citrate monohydrate (102004S) was obtained from VWR International (Radnor, PA, USA). Potassium hydroxide (105033) was bought from Merck (Merck KGaA, Darmstadt, Germany). Recombinantly produced chymosin (CHY-MAX Plus, lot no. 3642156) was obtained from Chr. Hansen Holding A/S (Hørsholm, Denmark).

Ultrapure water (MilliQ system, Merck KGaA, Darmstadt, Germany) was used for all experiments. Skim milk (0.1% fat, Melkan, Cooperatieve Inkoopvereniging Superunie B.A, Beesd, Netherlands), was purchased at a local grocery store.

### Preparation of Artificial Casein Micelles

Artificial casein micelles (ACM) were prepared by concentrating a dilute solution containing Mg, PO₄, Ca, citrate and caseinate in the ratios similar to those in which they occur in bovine milk (Table 1):

TABLE 1
Mineral concentrations in bovine milk²

| Constituents | Concentration (mmol kg⁻¹) |
|---|---|
| Calcium | 26-31 |
| Magnesium | 4-6 |
| Inorganic phosphate | 19-23 |
| Total phosphorus | 30-32 |
| Citrate | 7-11 |
| Sodium | 17-28 |
| Potassium | 31-43 |
| Chloride | 22-34 |

Solutions with different initial concentrations were prepared, as listed in Table 2, to explore the influence of the concentration of the starting solution:

TABLE 2
Starting composition and pH of samples prepared at a heating
temperature of 52° C. The concentration factor is
the factor required for the solutions to attain 30 mM Ca, 22 mM
PO₄, 5 mM Mg, 9 mM citrate and 25.6 g L − 1 casein.

Concentration factor (—)

| | | | | |
|---|---|---|---|---|
| 3 | 4.3 | 6 | 10 | 30 |

| | |
|---|---|
| Material | Concentration (g L⁻¹) |

| | | | | | |
|---|---|---|---|---|---|
| Na—CN | 9.459 | 6.622 | 4.730 | 2.838 | 0.946 |
| CaCl₂ | 1.110 | 0.777 | 0.555 | 0.333 | 0.111 |
| KH₂PO₄ | 0.998 | 0.699 | 0.499 | 0.299 | 0.100 |
| MgCl₂ | 0.159 | 0.111 | 0.079 | 0.048 | 0.016 |
| Citric acid | 0.576 | 0.403 | 0.288 | 0.173 | 0.058 |
| Initial pH | 7.25 | 7.25 | 7.35 | 7.35 | 7.55 |

The corresponding amounts of MgCl₂, KH₂PO₄ and citric acid were weighed on an analytical balance (EX224 Explorer Analytical, Ohaus Corporation, NJ, USA) and added to a Schott bottle containing 75% of the corresponding volume of ultrapure water. The weighing boats were rinsed into the Schott bottle, and the remaining volume was added. CaCl₂) granules were then added to the solution while it was being stirred. The pH was adjusted to 6.3-6.7 using 1 M NaOH. Afterwards, sodium caseinate was added to the solution and it was stirred on a heating plate (C-MAG HS 7, IKA-Werke Gmbh & CO. KG, Staufen, Germany) set to 70° C. for 30 minutes. The solution was then left to cool to room temperature for an additional 30 minutes of stirring until the sodium caseinate was dissolved. Finally, the pH of the solution was adjusted to the corresponding initial pH values listed in Table 2 using 1 M NaOH. The initial pH values were determined in preliminary experiments to ensure the pH after evaporation was ≈6.7. Samples were prepared in duplicate, except for evaporation rates of 655 and 1012 mL h⁻¹. After concentration, the formed ACM constituted 30 mM Ca, 22 mM PO₄, 5 mM Mg, 9 mM citrate and 25.6 g L⁻¹ casein. The resulting concentrate is further referred to as “VE-ACM” (Vacuum Evaporation Artificial Casein Micelles).

Vacuum evaporation was performed using a rotary evaporator (RC 900, KNF Holding AG, Sursee, Switzerland). A vacuum pump system (SC 920 G, KNF Holding AG, Sursee, Switzerland) decreased the pressure to 62 mBar, corresponding to a water boiling point of 37° C. (Wagner & Kretzschmar, 2008). A cryo-compact circulator (CF40, Julabo GmbH, Seelbach, Germany) was set to 4° C. and connected to a condensor. The solutions were carefully poured into an indented evaporation flask (powder flask 514-74200-00, Heidolph Instruments GmbH & CO. KG, Schwabach, Germany) wetted with ultrapure water. The rim of the evaporation flask was covered with a thin layer of silicone grease. After evaporation, the volume and pH of the sample were recorded. The Vacuum Evaporation Artificial Casein Micelles (VE-ACM) were stored at 4° C. until further analysis.

In a second set of experiments, the influence of the preparation rate was investigated by concentrating solutions with equal initial concentrations at different evaporation rates, achieved by setting the temperature of the water bath, further referred to as heating temperature, in a range from 46 to 80° C.

Artificial casein micelles (“ACMs”) were also prepared for comparison to the process of the present invention (Schmidt ACM; “S-ACM”), via the methods according to Schmidt et al. (1977) 1. Sodium caseinate was dissolved in water to a protein concentration of 64.0 g L⁻¹ by stirring at 60° C. for 30 minutes and subsequently adjusted to pH 8.00 with 1M NaOH. Three salt solutions were prepared: solution I contained 445 mM CaCl₂) and 75 mM MgCl₂ adjusted to pH 7.25 with 0.1M HCl, solution II contained 165 mM KH₂PO₄ and 165 mM Na₂HPO₄ adjusted to pH 7.25 with 1M NaOH, and solution III contained 135 mM C₆H₈O₇ adjusted to pH 7.25 with 1M KOH. The caseinate solution (60 mL) and the salt solutions (10 mL each) were carefully pumped into a jacketed glass vessel at 37° C. containing a starting volume of 60 mL water in 60 minutes to reach final concentrations of 30 mM calcium, 22 mM phosphate, 9 mM citrate, 5 mM magnesium, and 25.6 g L⁻¹ casein. The solution was continuously and vigorously stirred using a magnetic stirrer.

Controlled addition of the solutions was achieved by using syringe pumps (Harvard PHD2000, Harvard Apparatus, Holliston, MA, USA and ProSense NE-1600, ProSense B.V., Oosterhout, Netherlands). The pH decreased gradually from about 7.25 to 6.70 during preparation to mimic the gradual pH decrease during ACM preparation through vacuum evaporation and membrane processes as a result of the release of protons due to the formation of calcium phosphate nanoclusters. The final pH was adjusted, if necessary, to pH 6.70 with 1M NaOH. S-ACM samples were prepared in triplicate.

Forward osmosis (FO) was conducted with a forward osmosis module (Aquaporin Inside® HFFO®2, Aquaporin A/S, Kongens Lyngby, Denmark) with an active membrane area of 2.3 m². Feed solutions were recircled through the lumen at a flow rate of 60 L h⁻¹ and draw solutions were passed through the shell side at 10 L h⁻¹ in single-pass mode by means of two gear pumps. Feed and draw solutions were ran in counter-current flow. Feed solutions with an initial volume of 6 L were concentrated to about 1 L (c_{f}=6). The concentration time was controlled between 28 and 60 min by adjusting the concentration of the draw solution between 0.20 to 0.12M NaCl, respectively. Feed solutions were continuously stirred on a stirring plate and their temperature was controlled at 37° C. during concentration. If necessary, the pH of the ACM was adjusted to 6.70 with 1M NaOH after concentration. FO-ACM_{t=60 min} were prepared in triplicate and the rest of the FO-ACM (28 min, 38 min) in singlicate. The Forward Osmosis Artificial Casein Micelles (FO-ACM) were stored at 4° C. for at least 12 h until analysis.

Reverse osmosis (RO) was conducted with a CUBE80-VA cross-flow laboratory filtration unit (SIMA-Tec® GmbH, Schwalmtal, Germany) equipped with a membrane cell with an active membrane area of 85 cm². A flat sheet polyamide RO membrane (TRISEP® ACM2, MANN+HUMMEL Water & Fluid Solutions GmbH, Wiesbaden, Germany) was installed in the membrane cell. Feed solutions with an initial volume of 3.6 L were pumped through the membrane cell at a rate of 30 L h⁻¹. A pressure of on average 33 bar was applied and the temperature was maintained at 37° C. by the temperature control unit. Filtrations were continued for about 22 h until 3 L permeate was collected (c_{f}=6). To avoid microbial spoilage during an experiment, 0.02% (v/v) sodium azide was added to the feed solutions. If necessary, the pH of the ACM was adjusted to 6.70 with 1M NaOH after concentration. The Reverse Osmosis Artificial Casein Micelles (RO-ACM) were prepared in duplicate and stored at 4° C. for at least 12 h until analysis.

### Particle Size Analysis

The mean diameter and polydispersity index (PDI) of ACM were measured by using dynamic light scattering (Zetasizer Ultra, Mavern Panalytical Ltd, Worcestershire, UK). The samples were measured at 25° C. with a refractive index of 1.57. Samples were diluted 50- or 100-fold in simulated milk ultrafiltrate (SMUF; prepared according to Dumpler et al. (2017))³ in a DTS0012 square polystyrene cuvette and measured in duplicate. Each measurement consisted of five submeasurements. The ACM were stored for at least 24 hours prior to the measurement. Results are shown as the Z-average±SD and the corresponding PDI±SD, both of which were obtained from the ZSXplorer software (version 2.3.1.4).

### Scanning Electron Microscopy

Micelle morphology was investigated with scanning electron microscopy (SEM) by using a Magellan 400 microscope (FEI Company, Hillsboro, OR, USA). One drop of sample was pipetted onto 12 mm poly-L-lysine glass slides (Corning Inc., Corning, NY, USA) and left to adhere for 30 minutes. Subsequently, the glass slides were washed twice with SMUF and fixated with 2.5% glutaraldehyde in phosphate/citrate buffer at pH 7.2 for one hour after removal of the SMUF. Afterwards, the fixative was removed and the glass slides were washed six times with SMUF. Samples were then fixated with a 1% osmium tetroxide solution for one hour. The fixative was removed again and the slides were washed thrice with water and dehydrated using a graded ethanol series (5 minutes 30%, 5 minutes 50%, 5 minutes 70%, 5 minutes 80%, 5 minutes 90%, 5 minutes 96%, 10 minutes 100%, and another 10 minutes 100% EtOH). Subsequently, the samples were critical point dried with CO₂ using a Leica EM CPD 300 (Leica Biosystems GmbH, Nussloch, Germany). The sputter coated slides were affixed to an aluminium specimen stub using carbon adhesive tabs. Mounted specimens were coated with a 12 nm tungsten layer in a Leica EM SCD 500 sputter coater. Images were taken at 100,000× magnification.

### Ultracentrifugation

ACM were ultracentrifuged to obtain a serum phase and pellet for further analysis. Prior to the ultracentrifugation, the ACM sample tubes were taken out of the fridge and acclimated to room temperature for 1 hour. Samples were centrifuged for 1 hour at 20° C. and 100,000×g in a Beckman Coulter Optima XE-90 ultracentrifuge equipped with a 70Ti rotor (Beckman Coulter Inc., Woerden, Netherlands). Samples were centrifuged in duplicate. Using a transfer pipette, approximately half of the supernatant was collected and combined with the supernatant of the corresponding duplicate.

### Hydration

The remaining supernatant in the ultracentrifugation tubes was discarded and the tubes were placed upside-down in a tube rack for one hour to let excess liquid drop out. The pellet was transferred and weighed into aluminium pans. The pans were placed in an oven (E28, Binder GmbH, Tuttlingen, Germany) at 105° C. for minimum 48 hours. The pans with the dried pellets were weighed immediately after removal from the oven. The apparent casein micelle hydration was calculated according to Huppertz et al. (2017)⁴ by dividing the moisture content of the pellet by the dry matter of the pellet.

### Casein Content and Composition Determination

The total casein content and composition of the casein fractions of the ACM and corresponding supernatants and of skim milk was determined based on the method described in Schubert et al. (2018)⁵ using Reversed-Phase High Performance Liquid Chromatography (RP-HPLC) (Thermo Ultimate 3000 system, Thermo Fisher Scientific Inc., Waltham, USA). ACM were diluted 6-fold and the supernatants 3-fold in a buffer solution containing 6 M guanidine hydrochloride, 0.02 M L-dithiotreitol and 0.005 M sodium basic tribasic dihydrate. A solvent gradient (Table 3) with a flow rate of 1 mL min-1 was used. Two eluents were used: eluent A consisted of 1% acetonitrile (ACN) and 0.1% trifluoroacetic acid (TFA) in ultrapure water and eluent B consisted of 1% ultrapure water and 0.072% TFA in ACN. The employed column was a VDSpher OptiBio PUR 300 C4-SE (VDS Optilab, Berlin, Germany). The column oven was set to 30° C. The sample injection volume was 10 μL and the detection wavelength was 214 nm. Samples were analyzed in duplicate and are expressed as mean±SD (g L⁻¹).

TABLE 3
Gradient eluent of eluents A and B for RP-HPLC.

| Time (min) | Eluent A (%) | Eluent B (%) |
|---|---|---|
| 0.0 | 28.0 | 72.0 |
| 21.5 | 37.6 | 62.4 |
| 22.5 | 37.6 | 62.4 |
| 26.0 | 46.0 | 54.0 |
| 28.0 | 100.0 | 0.0 |
| 29.0 | 100.0 | 0.0 |
| 30.0 | 28.0 | 72.0 |
| 35.0 | 28.0 | 72.0 |

### Analysis of Mineral Partitioning

The content of anions (chloride, phosphate, and citrate) in samples and their supernatants was analyzed by ion chromatography (IC). Samples were diluted 200-fold and supernatants were diluted 500-fold in water. Solutions were then analyzed on a Dionex ICS-6000 liquid chromatography system equipped with a 2 mm standard bore Dionex lonPac AS17-C column for anion analysis (Thermo Fisher Scientific B.V., Breda, Netherlands) at 30° C. with a flow rate of 0.25 mL min⁻¹ and injection volume of 5 μL. Gradient elution was conducted with KOH, first set to 5 mM for 10 minutes, followed by a linear increase to 40 mM within 15 minutes, an isocratic elution at 40 mM for 6 minutes, and a linear decrease to 5 mM in 5 minutes. A conductivity detector was used for peak detection. Samples were analyzed in duplicate.

The content of cations (calcium, phosphorus, magnesium, sodium, and potassium) in samples and their supernatants was analyzed by inductively coupled plasma optical emission spectrometry (ICP-OES) First, samples were prepared by microwave-assisted wet digestion. Approximately 0.5 mL sample was weighed and 10 mL aqua regia (7.5 mL HCl+2.5 mL HNO₃) and 1 mL H₂O₂ were added. Subsequently, the mixture was subjected to a temperature program in an ETHOS EASY microwave digestion system (Milestone Srl, Sorisole, Italy) according to Guimarães et al. (2021).⁶ The digested material was washed and diluted with water to a total volume of 100 mL. Samples were prepared in duplicate and analyzed in triplicate by using the operational conditions as specified in Guimarães et al. (2021).⁶ Elements were detected in radial view, unless stated otherwise, at the following wavelengths: Ca 393.366 nm, P 178.221 nm (axial), Mg 279.553 nm, Na 589.592 nm, K 766.490 nm.

All supernatant concentrations of ionic species were corrected for the excluded volume with a correction factor K calculated according to Pierre and Brule (1981):⁷

$$K = \frac{1000 - P ( 1 + W )}{1000}$$

where P is the measured micellar protein content in g L⁻¹ and W the measured micelle hydration in g water g⁻¹ dry matter.

The serum phase refers to the minerals that remained in the supernatant upon ultracentrifugation, whereas the concentration of minerals in the micellar phase was calculated as the difference between the total concentration and the serum concentration.

Coagulation with Chymosin and Rheological Characterisation

Rennet-induced coagulation was monitored through oscillatory rheometry. ACMs were adjusted to pH 6.3 by means of acidification below 10° C. with a 1:10 lactic acid solution in water. Subsequently, 0.04% (v/w) calcium chloride was added by means of a 4% (w/v) calcium chloride solution and the samples were heated to 30° C. while stirring. Samples were then renneted by adding 0.02% (v/w) chymosin and transferred to a rheometer (MCR 302, Anton Paar, Graz, Austria) equipped with a double-gap device (DG26.7). A strain amplitude of 0.001 and a frequency of 1 Hz were applied while the temperature was controlled by a Peltier element set to 30° C. Samples were analyzed in duplicate. The skim milk reference was diluted with SMUF to a casein concentration of 26 g L⁻¹, based on the original casein concentration determined with RP-HPLC.

### Results and Discussion

### Effects of Vacuum Evaporation on ACM Preparation Rate

To gain a better understanding of the effects of the rate of preparation on the formation of ACMs as a function of concentration of precursor solutions via the vacuum evaporation process, the evaporation speed at various heating temperatures was characterized, as well as the temperature of the sample being concentrated. A fixed volume of water (500 mL) was evaporated at different speeds by adjusting to the heating temperature. The temperature of the concentrate was controlled by evaporating at 62 mBar, which corresponds to a boiling point of 37° C. for water. The temperature inside the evaporation flask remained between 37° C. and 40° C. throughout the evaporation process (Table 4):

TABLE 4
Temperature (° C.) of the concentrate inside the
evaporation flask during vacuum evaporation at 62
mBar at heating temperatures of 50, 55, 60 and 65° C.

Heating temperature (° C.)

| | | | | |
|---|---|---|---|---|
| Time | 50 | 55 | 60 | 65 |

| | |
|---|---|
| (min) | Sample temperature (° C.) |

| | | | | |
|---|---|---|---|---|
| 0 | 21.3 ± 0.5 | 22.2 ± 0.3 | 21.5 ± 0.7 | 21.7 ± 1.1 |
| 30 | 38.2 ± 0.1 | 38.9 ± <0.1 | 39.7 ± 0.4 | 39.8 ± 1.1 |
| 60 | 38.4 ± 0.3 | 39.1 ± 0.1 | 39.5 ± 0.5 | 40.2 ± 0.4 |
| 70 | 38.2 ± 0.2 | 39.0 ± 0.8 | 40.1 ± 0.7 | 39.1 ± 1.3 |
| 90 | 38.1 ± <0.1 | — | — | — |
| 130 | 37.5 ± 0.7 | — | — | — |
| 160 | 37.9 ± 0.6 | — | — | — |

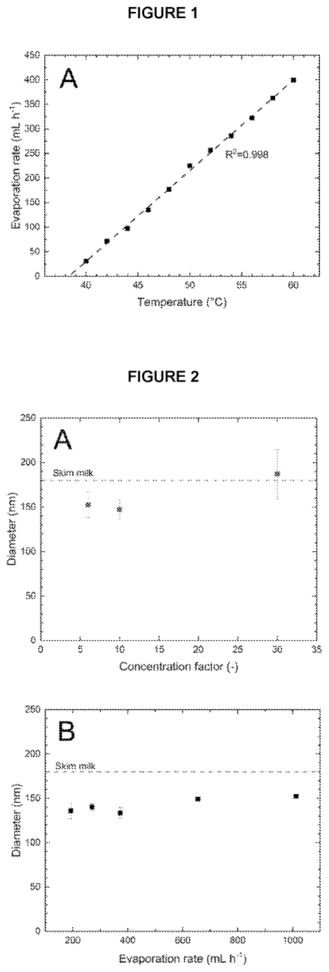

The evaporation rate increased linearly (R2=0.998) from 31.0 mL h⁻¹ at 40° C. to 399.5 mL h⁻¹ at 60° C. (FIG. 1) according to the following relation:

Evaporation rate (mL h⁻¹)=18.47×T−708.58

Theoretically, the x-axis intercept should be at 37° C., which is equal to the boiling point of water at 62 mBar. By calculation, the x-axis intercept is slightly higher (38.4° C.), which is possibly caused by inaccuracies in the pressure control of the vacuum evaporator. The linear increase of the evaporation rate is expected to continue up to a heating temperature of around 100° C., at which point the boiling point of water at atmospheric pressure is reached and the heating temperature cannot be increased further. Practically, the heating temperature is already approaching its maximum at 80° C., where the connected cooling bath cannot maintain the temperature of the cooling water for the condensor to condense the solvent vapor. However, this may be ameliorated via the use of a more powerful cooling bath.

### ACM Properties

In a first series of experiments (Series 1), VE-ACM were prepared by vacuum evaporation of solutions containing minerals and casein in the ratios found in bovine milk at 62 mBar (=37° C.). The initial concentration of these solutions was varied to investigate the influence of the initial concentration of the solution on the formation of ACM. Furthermore, solutions of a fixed initial concentration were evaporated at various evaporation rates to assess the influence of the evaporation rate. An overview of the prepared Series 1 samples is listed in Table 5 and more details on volume and pH after concentration and evaporation time can be found in Tables 6 and 7.

TABLE 5
Overview of VE-ACM prepared with different concentration
factors and evaporation rates.

| | | | | |
|---|---|---|---|---|
| Concentration | Initial | Heating | Target | Evaporation |
| factor | volume | temperature | volume | rate |
| (—) | (mL) | (° C.) | (mL) | (mL h⁻¹) |

Varying initial concentrations

| | | | | |
|---|---|---|---|---|
| 3* | 750 | 52 | 250 | 353 |
| 4.3* | 750 | 52 | 175 | 353 |
| 6 | 750 | 52 | 125 | 353 |
| 10 | 750 | 52 | 75 | 353 |
| 30 | 1000 | 52 | 33 | 353 |

Varying evaporation rates

| | | | | |
|---|---|---|---|---|
| 10 | 750 | 46 | 75 | 193 |
| 10 | 750 | 50 | 75 | 270 |
| 10 | 750 | 54 | 75 | 373 |
| 10 | 750 | 65 | 75 | 655 |
| 10 | 750 | 80 | 75 | 1012 |

*visually unstable against calcium phosphate precipitation

TABLE 6
Obtained volumes and pH values after vacuum evaporation for
VE-ACM concentrated with increasing concentration factors.

| Concentration factor (—) | 3 | 4.3 | 6 | | 10 | | 30 | |
|---|---|---|---|---|---|---|---|---|
| Replicate | 1 | 2 | 1 | 2 | 1 | 2 | 1 | 2 |
| Initial volume (mL) | 750 | 750 | 750 | 750 | 750 | 750 | 1000 | 1000 |
| Target end volume (mL) | 250 | 175 | 125 | 125 | 75 | 75 | 33.33 | 33.33 |
| Actual end volume (mL) | 260 | 177 | 122 | 128 | 71 | 75 | 36 | 32 |
| Initial pH | 7.24 | 7.25 | 7.35 | 7.35 | 7.34 | 7.36 | 7.58 | 7.58 |
| End pH | n.d. | n.d. | 6.81 | 6.79 | 6.66 | 6.74 | 6.79 | 6.76 |
| Evaporation time (min) | n.d. | n.d. | n.d. | n.d. | 115 | n.d. | 165 | n.d. |
| Evaporation rate (mL h⁻¹) | n.d. | n.d. | n.d. | n.d. | 353 | n.d. | 351 | n.d. |

TABLE 7
Obtained volumes and pH values after vacuum evaporation for VE-ACM concentrated
with increasing heating temperature (=increasing evaporation rate).

| Heating temperature (° C.) | 46 | | 50 | | 54 | | 65 | 80 |
|---|---|---|---|---|---|---|---|---|
| Replicate | 1 | 2 | 1 | 2 | 1 | 2 | 1 | 1 |
| Inital volume (mL) | 750 | 750 | 750 | 750 | 750 | 750 | 750 | 750 |
| Target end volume (mL) | 75 | 75 | 75 | 75 | 75 | 75 | 75 | 75 |
| Actual end volume (mL) | 76 | 74.5 | 77 | 74 | 79 | 71 | 73 | 76 |
| Initial pH (—) | 7.36 | 7.35 | 7.36 | 7.39 | 7.37 | 7.37 | 7.34 | 7.31 |
| End pH (—) | 6.79 | 6.71 | 6.79 | 6.75 | 6.91 | 6.84 | 6.79 | 6.77 |
| Evaporation time (min) | n.d. | 210 | n.d. | 150 | n.d. | 109 | 62 | 41 |
| Evaporation rate (mL h⁻¹) | n.d. | 193 | n.d. | 270 | n.d. | 373 | 655 | 1012 |

According to the observations made, it appeared that micelle-like particles formed during concentration of the dilute solutions, due to the transition to a white and opaque solution. During vacuum evaporation, water evaporated from the solutions and the effective concentration of the solutes in the concentrate increased as a result, which must have caused calcium and phosphate to cluster and interact with the present caseins, thereby forming micellar structures. The mean hydrodynamic diameter of these micellar structures in samples prepared with different initial concentrations is shown in FIG. 2a. The initial concentrations corresponded to concentration factors of 3×, 4.3×, 6×, 10× and 30× to reach the approximate composition of bovine milk. The evaporation rate was kept constant at 353 mL h⁻¹ (=52° C. heating temperature). Initial solutions to be concentrated with concentration factors of 3× and 4.3× were not analyzed since sedimentation of the components was observed before evaporation. It is likely that this sediment consisted of HAP as a consequence of uncontrolled nucleation of CaP and subsequent transformation into the thermodynamically more stable phase. Thus, these initial solutions were presumably already supersaturated with respect to CaP and crystallization occurred prior to evaporation. The mean micelle diameter of VE-ACM produced with different concentration factors appeared to decrease slightly from 152.6±14.3 to 147.1±10.6 nm upon increasing the concentration factor from 6× to 10× and then seemed to increase to 187.3±28.1 nm at a factor of 30×. FIG. 2b depicts the mean diameter of VE-ACM that were concentrated 10-fold while varying the evaporation rate between 193 to 1012 mL h⁻¹. It appears that the micelle diameter follows a slight increase with the applied evaporation rate from 136.0±9.0 nm at 193 mL h⁻¹ to 152.4 nm at 1012 mL h⁻¹, where the size of the ACM prepared at 373 mL h⁻¹ (133.6±3.5 nm) fell out of this trend. Bovine casein micelles in skim milk were measured to be 179.9 nm in diameter. Therefore, the sample concentrated 30-fold had a similar size as natural casein micelles, whereas the VE-ACM prepared with concentration factors of 6× and 10× and with different evaporation rates were smaller, but still fit in the expected range for bovine casein micelles between 50 and 500 nm.

In a second series of experiments (Series 2), VE-ACM were prepared using the same protocol as for Series 1, at a vacuum pressure of 63 mBar, (corresponding to a boiling point of 37° C.) and with a fixed concentration factor of 6 (900 mL initial solution concentrated to 150 mL) and varied the heating bath temperature of the rotary evaporator from 53° C. to 80° C. in order to vary the rate of evaporation and thus the preparation time of the VE-ACM for each batch. An overview of the evaporation rates and preparation times arising from the heating bath temperatures applied to each ACM batch is provided in Table 8:

TABLE 8
Heating temperatures used during the preparation of VE-ACM
and the resulting preparation times and evaporation rates.

| Heating temperature | Preparation time | Evaporation rate |
|---|---|---|
| (° C.) | (min) | (mL/h) |
| 53 | 105 | 7.2 |
| 58 | 80 | 9.4 |
| 63 | 60 | 12.6 |
| 73 | 46 | 16.5 |
| 80 | 41 | 18.5 |

These ACM prepared through vacuum evaporation were compared to ACM prepared according to the method of Schmidt et al. (1977) 1 hereinafter referred to as the “Schmidt method” (S-ACM). The Schmidt method involves micelle formation over a period of an hour at 37° C. The VE-ACM prepared at a heating temperature of 63° C. were formed in 60 minutes and are therefore most directly comparable to S-ACM. Since the boiling point of water is at 37° C. during all our evaporation experiments, it is reasonable to assume that the temperature at which micelle formation occurred was similar to the Schmidt method.

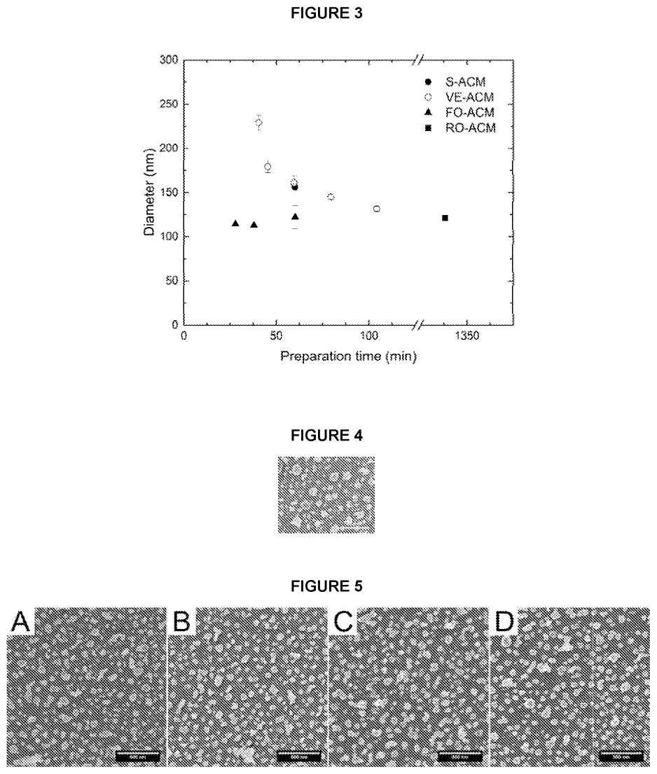

The varying evaporation rates and preparation times effects on the speed of the micelle formation did influence the size of the VE-ACMs produced. Faster formation of micelles (higher heating temperatures/lower preparation times/higher evaporation rates) yielded larger micelles, whereas slower formation of micelles yielded smaller micelles (FIG. 3). S-ACM were 156.3±3.5 nm in diameter, which is very similar to the VE-ACM at an equal preparation time (161.0±7.6 nm). Meanwhile, the ACM prepared via forward osmosis (FO-ACM) and reverse osmosis (RO-ACM) were slightly smaller than those prepared via vacuum evaporation (VE-ACM) and the method of Schmidt (S-ACM). RO-ACM fitted in the trend of decreasing micelle size at increased preparation times found for ACM prepared through vacuum evaporation (VE-ACM). FO-ACM differed in this respect, which may be attributed to large surface (2.3 m²) over which micelle formation occurred in the forward osmosis experiments (FIG. 3). FO-ACM prepared in 60 minutes were significantly (p<0.0001) smaller at 121.9±12.9 nm. As noted above, the diameter of VE-ACM strongly depended on the preparation time, where increased preparation times yielded smaller micelles. In accordance with this theory, RO-ACM were significantly (p<0.0001) smaller than S-ACM and VE-ACM due to the long preparation time of 22 hours. The size of FO-ACM did not seem to depend on the preparation time.

Scanning Electron Microscope (SEM) images of bovine skim milk (FIG. 4) and the Series 2 experiments show that S-ACM produced by the Schmidt method (FIG. 5a), VE-ACM produced by the process of the present invention via vacuum evaporation (FIG. 5b), FO-ACM produced by the process of the present invention via forward osmosis (FIG. 5c), each with a preparation time of 1 hour, and RO-ACM produced by the process of the present invention via reverse osmosis (FIG. 5d) with a preparation time of 22 hours, are extremely similar on the nanoscale in terms of micelle sizes, shape irregularities and size polydispersities, and reasonably similar to the micelles found in bovine skim milk.

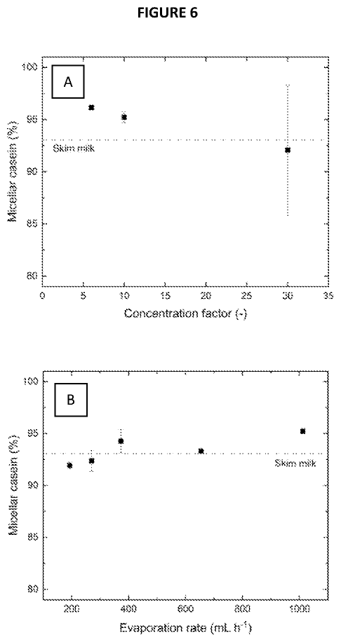

The micellar casein content of the VE-ACM (Series 1) prepared with different concentration factors is depicted in FIG. 6a. Total concentrations of casein in the VE-ACM and corresponding concentrations are listed in Table 9:

TABLE 9
Total casein content (g L⁻¹) and serum casein content (g
L⁻¹) with corresponding percentages of micellar casein for VE-
ACM prepared with different concentration factors and evaporation rates.

| | | |
|---|---|---|
| Total casein | Non-sedimentable | Micellar casein |
| content (g L−¹) | casein (g L⁻¹) | (%) |

Concentration factor (—)

| | | | |
|---|---|---|---|
| 6 | 28.4 ± 1.1 | 1.1 ± 0.1 | 96.2 ± 0.2 |
| 10 | 29.4 ± 0.7 | 1.4 ± 0.2 | 95.2 ± 0.5 |
| 30 | 22.9 ± 0.4 | 1.8 ± 1.2 | 92.1 ± 6.2 |

Evaporation rate (mL h⁻¹)

| | | | |
|---|---|---|---|
| 193 | 26.6 ± 2.9 | 2.2 ± 0.2 | 91.9 ± 0.3 |
| 270 | 26.8 ± 3.3 | 2.0 ± 0.1 | 92.4 ± 1.0 |
| 373 | 30.1 ± 3.4 | 1.7 ± 0.1 | 94.3 ± 1.1 |
| 655 | 24.0 | 1.6 | 93.3 |
| 1012 | 22.8 | 1.1 | 95.2 |

The micellar casein increased from 95.2±0.5% to 96.2±0.2% upon change of the concentration factor from 10× to 6×. The sample concentrated 30× had the lowest amount of micellar casein with 92.1±6.2%. The micellar casein content of VE-ACM is comparable to that of skim milk, where 93.1% of casein was in the micellar phase. The micellar casein content for samples prepared with increasing evaporation rates is shown in FIG. 6b. The micellar casein content increased for the samples prepared from 193 mL h⁻¹ (91.9±0.3%) to 373 mL h⁻¹ (94.3±1.1%), but then slightly decreased to 93.3% for the samples prepared at 655 mL h⁻¹. The highest micellar casein content was observed for the VE-ACM prepared at an evaporation rate of 1012 mL h⁻¹ with 95.2%. The micellar protein content of the VE-ACM was in good agreement with the amounts observed in skim milk (93.1%). It appears that the micellar casein content was not considerably affected by the evaporation rate.

Similarly with the Series 2 VE-ACM experiments, compared to S-ACM, a larger proportion of the caseins in VE-ACM prepared in accordance with the present invention were present in the micellar phase and actually formed micelles (Table 10), whilst the preparation time did not seem to significantly influence the level of micellar casein observed for these VE-ACM.

TABLE 10
Total protein concentrations and percentages of casein in the
micellar phase of ACM prepared with the Schmidt method (S-ACM),
prepared through vacuum evaporation (VE-ACM) at different heating
temperatures (Series 2), prepared via forward osmosis (FO-ACM)
at formation times of 28, 38, and 60 minutes, and prepared via
reverse osmosis (RO-ACM) at a formation time of 22 hours.

| | Total casein (g/L) | Micellar casein (%) |
|---|---|---|
| FO-ACM_{t=28 min} | 25.2 ± 0.4 | 91.4 ± <0.1 |
| FO-ACM_{t=38 min} | 28.1 ± 0.1 | 91.9 ± 0.1 |
| VE-ACM_{t=41 min} | 25.9 ± 0.2 | 95.1 ± <0.1 |
| VE-ACM_{t=46 min} | 26.1 ± 0.9 | 95.9 ± 0.1 |
| S-ACM_{t=60 min} | 25.0 ± 0.2 | 91.8 ± 0.6 |
| VE-ACM_{t=60 min} | 26.0 ± 0.3 | 95.1 ± 0.4 |
| FO-ACM_{t=60 min} | 27.4 ± 2.7 | 95.1 ± 1.4 |
| VE-ACM_{t=80 min} | 25.6 ± 0.7 | 95.8 ± <0.1 |
| VE-ACM_{t=105 min} | 26.2 ± 0.9 | 96.2 ± <0.1 |
| RO-ACM_{t=22 h} | 23.0 ± 0.2 | 96.9 ± 1.6 |

The mineral partition of the samples was determined as described in Bijl et al. (2013).⁸ The results for ACM prepared with different concentration factors (Series 1) are shown in Table 11:

TABLE 11
Mineral partition of VE-ACM produced with
concentration factors of 6, 10 and 30.

| | Concentration factor (—) | | |
|---|---|---|---|
| Parameter | 6 | 10 | 30 |
| Total Ca (mM) | 32.5 ± 0.4 | 32.5 ± 1.8 | 30.2 ± 4.8 |
| Nonsedimentable Ca (mM) | 9.1 ± 0.2 | 10.4 ± 1.1 | 9.3 ± 1.1 |
| Micellar Ca (%) | 72.1 ± 1.1 | [symbol] | [symbol] |
| Micellar Ca (mM) | 23.4 ± 0.7 | [symbol] | 20.5 ± 4.4 |
| Micellar Ca/g micellar casein (mM) | 0.9 ± 0.1 | [symbol] | 1.0 ± 0.3 |
| Total Mg (mM) | 4.8 ± <0.1 | [symbol] | 4.2 ± 0.7 |
| Nonsedimentable Mg (mM) | 2.9 ± 0.2 | 3.1 ± 0.2 | [symbol] |
| Micellar Mg (%) | 40.3 ± 3.8 | [symbol] | 32.2 ± 3.5 |
| Micellar Mg (mM) | 1.9 ± 0.2 | [symbol] | 1.4 ± 0.4 |
| Micellar Mg/g micellar casein (mM) | 0.07 ± <0.1 | 0.06 ± <0.1 | 0.07 ± <0.1 |
| Total P (mM) | 29.3 ± 0.1 | [symbol] | 26.5 ± 4.1 |
| Total inorganic PO₄ (mM) | 24.6 ± 0.6 | 24.2 ± 1.2 | 23.6 ± 1.3 |
| Nonsedimentable P (mM) | 11.0 ± 0.6 | 12.1 ± 1.1 | 11.2 ± 1.5 |
| Nonsedimentable inorganic PO₄ (mM) | 11.0 ± 0.2 | [symbol] | 11.2 ± 1.1 |
| Micellar inorganic PO₄ (%) | 55.5 ± 0.7 | 49.8 ± 2.4 | 52.6 ± 2.2 |
| Micellar inorganic PO₄ (mM) | 13.7 ± 0.5 | 12.0 ± 0.2 | 12.4 ± 0.3 |
| Organic ester PO₄ (mM) | 0.1 ± 0.5 | −0.1 ± <0.1 | [symbol] |
| Micellar organic PO₄ (mM) | 18.2 ± 1.1 | [symbol] | 15.4 ± 2.5 |
| Micellar P/g of micellar casein (mM) | 0.7 ± 0.1 | [symbol] | 0.7 ± 0.2 |
| Total citrate (mM) | 11.1 ± 0.2 | [symbol] | [symbol] |
| Nonsedimentable citrate (mM) | 8.5 ± 0.1 | [symbol] | [symbol] |
| Micellar citrate (mM) | 2.6 ± 0.2 | 1.1 ± 0.1 | 2.4 ± 0.4 |
| Micellar citrate (%) | 23.4 ± 1.8 | [symbol] | 22.4 ± 5.0 |
| Total Na (mM) | 75.3 ± 1.0 | 76.9 ± 4.2 | 66.1 ± 11.6 |
| Total K (mM) | 22.9 ± 0.4 | 24.2 ± 1.4 | [symbol] |
| Total Cl (mM) | 73.2 ± 1.0 | 77.7 ± 4.8 | 72.3 ± 3.6 |

[symbol]

indicates data missing or illegible when filed

VE-ACM with concentration factors of 3 and 4.3 were not analyzed. The amount of micellar calcium ranged between 68.1±2.0% (10× concentration) to 72.1±1.1% (6× concentration). The mineral partitioning of samples prepared with different evaporation rates is shown in Table 12:

TABLE 12
Mineral partition of VE-ACM produced with evaporation
rates between 193 and 1012 mL h⁻¹.

| | Evaporation rate (mL h⁻¹) | | | | |
|---|---|---|---|---|---|
| Parameter | 193 | 270 | 373 | 655 | 1012 |
| Total Ca (mM) | 31.3 ± 1.0 | 32.1 ± 1.3 | 31.5 ± 2.4 | 28.9 | 25.3 |
| Nonsedimentable Ca (mM) | 8.9 ± 0.5 | 8.5 ± 0.5 | 10.1 ± 0.4 | 7.8 | 7.6 |
| Micellar Ca (%) | 71.5 ± 0.5 | 73.6 ± 0.3 | 67.9 ± 2.7 | 73.2 | 69.8 |
| Micellar Ca (mM) | 22.4 ± 0.2 | 23.6 ± 1.2 | 21.4 ± 2.6 | 21.2 | 17.7 |
| Micellar Ca/g of casein (mM) | 0.8 ± <0.1 | 1.0 ± 0.1 | 0.8 ± <0.1 | 0.9 | 0.8 |
| Total Mg (mM) | 4.8 ± 0.1 | 4.9 ± 0.2 | 4.9 ± 0.4 | 4.6 | 4.2 |
| Nonsedimentable Mg (mM) | 2.7 ± 0.1 | 2.7 ± 0.2 | 2.7 ± 0.3 | 2.8 | 2.6 |
| Micellar Mg (%) | 43.2 ± 0.1 | 44.4 ± 1.4 | 45.3 ± 2.8 | 39.4 | 37.0 |
| Micellar Mg (mM) | 2.1 ± <0.1 | 2.2 ± 0.2 | 2.2 ± <0.1 | 1.8 | 1.5 |
| Micellar Mg/g micellar casein (mM) | 0.08 ± 0.01 | 0.09 ± 0.01 | 0.08 ± 0.01 | 0.08 | 0.07 |
| Total P (mM) | 28.7 ± 1.0 | 29.1 ± 1.2 | 29.1 ± 2.1 | 27.9 | 25.0 |
| Total inorganic PO₄ (mM) | 23.0 ± 1.0 | 24.0 ± 1.5 | 24.2 ± 2.0 | 21.5 | 21.4 |
| Nonsedimentable P (mM) | 10.5 ± 0.5 | 10.3 ± 0.5 | 10.3 ± 0.8 | 10.0 | 9.5 |
| Nonsedimentable inorganic PO₄ (mM) | 11.5 ± 0.4 | 10.6 ± 0.7 | 10.5 ± 0.6 | 10.8 | 10.4 |
| Micellar inorganic PO₄ (%) | 49.9 ± 4.5 | 55.9 ± 0.6 | 56.7 ± 0.8 | 49.9 | 51.5 |
| Micellar inorganic PO₄ (mM) | 11.5 ± 1.6 | 13.4 ± 0.7 | 13.8 ± 1.4 | 10.7 | 11.0 |
| Organic ester PO₄ (mM) | −1.0 ± 0.3 | −0.2 ± 0.4 | −0.1 ± 0.2 | −0.8 | −0.9 |
| Micellar organic PO₄ (mM) | 19.2 ± 0.8 | 19.0 ± 1.4 | 18.9 ± 0.9 | 18.7 | 16.4 |
| Micellar P/g of micellar casein (mM) | 0.7 ± <0.1 | 0.8 ± 0.1 | 0.7 ± 0.1 | 0.8 | 0.7 |
| Total citrate (mM) | 7.0 ± 0.3 | 8.3 ± 0.7 | 7.8 ± 2.2 | 9.9 | 9.6 |
| Nonsedimentable citrate (mM) | 8.4 ± 0.1 | 8.4 ± 0.5 | 8.0 ± 1.1 | 8.7 | 8.6 |
| micellar citrate (mM) | −1.3 ± 0.2 | −0.2 ± 0.4 | −0.2 ± 1.5 | 1.2 | 1.0 |
| Micellar citrate (%) | −18.7 ± 3.3 | −2.2 ± 4.7 | −5.8 ± 20.7 | 12.1 | 10.6 |
| Total Na (mM) | 73.2 ± 2.5 | 77.6 ± 3.2 | 78.5 ± 5.6 | 72.0 | 71.2 |
| Total K (mM) | 22.5 ± 0.7 | 23.3 ± 1.5 | 23.6 ± 1.8 | 21.4 | 20.8 |
| Total Cl (mM) | 68.4 ± 2.3 | 72.0 ± 4.0 | 73.3 ± 5.7 | 69.1 | 67.3 |

VE-ACM prepared with different evaporation rates showed larger proportions of micellar calcium from 67.9±2.7% (373 mL h⁻¹) up to 73.6±0.3% (270 mL h⁻¹) compared to the ACM from the other set of experiments with 68.1±2.0%, although all samples had the same concentration factor. The micellar calcium of ACM produced via evaporation is slightly lower than the proportion found in in bovine skim milk with 72.5±4.3% (Bijl et al., 2013).⁸ The ACM produced by evaporation appear to absorb comparable amounts of minerals to ACM produced by the method of Schmidt, and to skim milk. It is apparent that despite variations in process conditions during evaporation, the micellar minerals remain relatively constant, presenting an opportunity to combine low concentration factors with high evaporation rates.

Similarly with the Series 2 experiments, the samples of ACMs prepared with the Schmidt method (S-ACM) and ACM prepared through vacuum evaporation (VE-ACM) were also very comparable in mineral composition (Table 13A):

TABLE 13A
Total concentration and proportion of this concentration present in the micellar phase.

| | | | | VE- | VE- | VE- |
|---|---|---|---|---|---|---|
| Parameter | S-ACM | VE-ACM_{t=105 min} | VE-ACM_{t=80 min} | ACM_{t=60 min} | ACM_{t=46 min} | ACM_{t=41 min} |
| Total Ca (mM) | 29.0 ± 0.2 | 28.6 ± 0.4 | 28.3 ± <0.1 | 28.6 ± 0.2 | 28.9 ± 0.1 | 28.5 ± 0.5 |
| Micellar Ca (%) | 71.5 ± 0.2 | 67.3 ± 0.3 | 69.5 ± 0.4 | 68.9 ± 0.9 | 70.1 ± 0.5 | 70.7 ± 0.6 |
| Total Mg (mM) | 4.6 ± 0.1 | 4.5 ± 0.1 | 4.5 ± <0.1 | 4.5 ± 0.1 | 4.4 ± <0.1 | 4.3 ± <0.1 |
| Micellar Mg (%) | 38.6 ± 0.9 | 33.9 ± 2.0 | 34.9 ± 1.7 | 34.4 ± 0.7 | 37.0 ± 0.2 | 36.4 ± 0.1 |
| Total P (mM) | 27.7 ± 0.3 | 27.8 ± 0.5 | 27.8 ± 0.4 | 27.5 ± 0.1 | 28.4 ± 0.1 | 27.5 ± 0.2 |
| Total inorganic PO₄ (mM) | 21.1 ± 0.3 | 21.2 ± <0.1 | 21.7 ± 0.4 | 21.5 ± 0.2 | 21.8 ± <0.1 | 19.7 ± 0.1 |
| Nonsedimentable P (mM) | 10.9 ± 0.1 | 11.8 ± 0.3 | 11.6 ± 0.1 | 11.6 ± 0.1 | 11.0 ± 0.2 | 10.1 ± 0.1 |
| Nonsed. inorganic PO₄ (mM) | 10.0 ± <0.1 | 11.5 ± <0.1 | 10.9 ± 0.2 | 11.1 ± <0.1 | 10.5 ± <0.1 | 9.9 ± <0.1 |
| Micellar inorganic PO₄ (%) | 52.6 ± 0.9 | 46.0 ± 0.1 | 49.9 ± 0.1 | 48.3 ± 0.5 | 52.1 ± <0.1 | 49.9 ± 0.3 |
| Total citrate (mM) | 10.1 ± 0.1 | 9.6 ± <0.1 | 9.9 ± 0.2 | 9.3 ± 0.1 | 9.7 ± <0.1 | 8.2 ± 0.1 |
| Micellar citrate (%) | 23.4 ± 1.5 | 19.9 ± 0.1 | 22.5 ± 0.3 | 17.8 ± 1.2 | 23.9 ± 0.1 | 12.0 ± 0.5 |
| Total Na (mM) | 55.7 ± 0.2 | 68.3 ± 1.2 | 70.0 ± 0.5 | 69.2 ± 0.5 | 68.4 ± 0.2 | 66.6 ± 0.9 |
| Total K (mM) | 37.9 ± 0.3 | 22.5 ± 0.7 | 22.2 ± 0.1 | 22.4 ± 0.5 | 21.6 ± 0.3 | 20.8 ± 0.1 |
| Total Cl (mM) | 68.0 ± 1.2 | 66.5 ± 0.1 | 69.2 ± 0.8 | 68.3 ± 1.3 | 68.3 ± 0.1 | 63.3 ± 0.1 |

The proportion of minerals in the micellar phase of VE-ACM (Series 2) prepared through evaporation was slightly lower than that of S-ACM, but the preparation time did not influence the proportions of micellar minerals substantially.

The proportion of minerals in the micellar phase of ACM prepared via forward osmosis (FO-ACM) and reverse osmosis (RO-ACM) was very similar to ACM prepared with vacuum evaporation (VE-ACM; Series 2) and via the Schmidt method (S-ACM), although ions diffused both from the feed to the draw solution (forward diffusion) and vice versa from the draw to the feed solution (reverse diffusion) during FO. This resulted in lower concentrations of calcium and potassium and higher concentrations of sodium and chloride in these samples (Table 13B). In the reverse osmosis preparation, the feed solution had an initial volume of 3.6 L and 3 L permeate was collected, in which no traces of casein or ions were found. This means that the target concentration factor was reached, and that the concentrations of casein and minerals in RO-ACM were slightly lower due to membrane fouling. A direct comparison of the total concentrations of casein and calcium (Ca), inorganic phosphorus (Pi) in the form of soluble phosphate, magnesium (Mg), citrate (Cit), sodium (Na), potassium (K), and chloride (CI) in these prepared samples is provided in Table 13C.

TABLE 13B
Total concentration and proportion of this concentration present in the micellar phase.

| Parameter | FO-ACM_{t=28 min} | FO-ACM_{t=38 min} | FO-ACM_{t=60 min} | RO-ACM_{t=22 h} |
|---|---|---|---|---|
| Total Ca (mM) | 23.4 ± 0.4 | 23.6 ± 0.3 | 25.6 ± 0.5 | 22.2 ± 0.2 |
| Micellar Ca (%) | 66.8 ± 0.8 | 68.6 ± 0.3 | 68.7 ± 3.1 | 68.4 ± 0.4 |
| Total Mg (mM) | 4.3 ± 0.1 | 4.3 ± <0.1 | 4.5 ± 0.1 | 3.8 ± <0.1 |
| Micellar Mg (%) | 33.1 ± 0.2 | 36.8 ± 0.8 | 35.9 ± 2.2 | 34.7 ± 0.7 |
| Total P (mM) | 25.7 ± 0.1 | 26.5 ± 0.4 | 26.4 ± 0.3 | 21.7 ± 0.2 |
| Total inorganic PO₄ (mM) | 22.3 ± 0.1 | 23.3 ± 0.1 | 22.2 ± 2.4 | 18.3 ± 0.7 |
| Nonsedimentable P (mM) | 11.6 ± <0.1 | 11.9 ± 0.1 | 11.5 ± 0.5 | 8.9 ± 0.2 |
| Nonsed. inorganic PO₄ (mM) | 13.5 ± <0.1 | 13.6 ± 0.1 | 12.3 ± 1.0 | 9.7 ± 0.2 |
| Micellar inorganic PO₄ (%) | 39.4 ± 0.2 | 41.7 ± 0.6 | 44.3 ± 3.2 | 47.3 ± 3.2 |
| Total citrate (mM) | 10.3 ± <0.1 | 10.6 ± <0.1 | 10.1 ± 1.0 | 8.5 ± 0.3 |
| Micellar citrate (%) | 16.2 ± 0.4 | 19.0 ± 0.8 | 19.2 ± 2.7 | 17.3 ± 4.8 |
| Total Na (mM) | 122.7 ± 4 | 124.8 ± 0.7 | 121.7 ± 2.6 | 63.6 ± 1.0 |
| Total K (mM) | 1.2 ± 0.1 | 0.9 ± 0.1 | 1.1 ± 0.6 | 15.9 ± 0.2 |
| Total Cl (mM) | 103.5 ± 0.6 | 101.5 ± 0.4 | 102.5 ± 1.4 | 56.6 ± 2.0 |

TABLE 13C
Total concentrations of casein and calcium (Ca), inorganic phosphorus (Pᵢ) in the form of soluble phosphate,
magnesium (Mg), citrate (Cit), sodium (Na), potassium (K), and chloride (Cl) in the prepared samples.

| Sample | Casein (g/L) | Ca (mM) | Pᵢ (mM) | Mg (mM) | Cit (mM) | Na (mM) | K (mM) | Cl (mM) |
|---|---|---|---|---|---|---|---|---|
| S-ACM_{t=60 min} | 25.4 ± 0.3 | 29.0 ± 0.2 | 21.1 ± 0.3 | 4.6 ± 0.1 | 10.1 ± 0.1 | 55.7 ± 0.2 | 37.9 ± 0.3 | 68.0 ± 1.2 |
| VE-ACM_{t=41 min} | 25.1 ± 0.1 | 28.5 ± 0.5 | 19.7 ± 0.1 | 4.3 ± <0.1 | 8.2 ± 0.1 | 66.6 ± 0.9 | 20.8 ± 0.1 | 63.3 ± 0.1 |
| VE-ACM_{t=46 min} | 25.8 ± <0.1 | 28.9 ± 0.1 | 21.8 ± <0.1 | 4.4 ± <0.1 | 9.7 ± <0.1 | 68.4 ± 0.2 | 21.6 ± 0.3 | 68.3 ± 0.1 |
| VE-ACM_{t=60 min} | 24.8 ± 1.2 | 28.6 ± 0.2 | 21.5 ± 0.2 | 4.5 ± 0.1 | 9.3 ± 0.1 | 69.2 ± 0.5 | 22.4 ± 0.5 | 68.3 ± 1.3 |
| VE-ACM_{t=80 min} | 26.4 ± 0.5 | 28.3 ± <0.1 | 21.7 ± 0.4 | 4.5 ± <0.1 | 9.9 ± 0.2 | 70.0 ± 0.5 | 22.2 ± 0.1 | 69.2 ± 0.8 |
| VE-ACM_{t=105 min} | 26.5 ± 0.2 | 28.6 ± 0.4 | 21.2 ± <0.1 | 4.5 ± 0.1 | 9.6 ± <0.1 | 68.3 ± 1.2 | 22.5 ± 0.7 | 66.5 ± 0.1 |
| FO-ACM_{t=28 min} | 25.2 ± 0.4 | 23.4 ± 0.4 | 22.3 ± 0.1 | 4.3 ± 0.1 | 10.3 ± <0.1 | 122.7 ± 4.0 | 1.2 ± 0.1 | 103.5 ± 0.6 |
| FO-ACM_{t=38 min} | 28.1 ± 0.1 | 23.6 ± 0.3 | 23.3 ± 0.1 | 4.3 ± <0.1 | 10.6 ± <0.1 | 124.8 ± 0.7 | 0.9 ± 0.1 | 101.5 ± 0.4 |
| FO-ACM_{t=60 min} | 27.4 ± 2.7 | 25.6 ± 0.5 | 22.2 ± 2.4 | 4.5 ± 0.1 | 10.1 ± 1.0 | 121.7 ± 2.6 | 1.1 ± 0.6 | 102.5 ± 1.4 |
| RO-ACM_{t=22 h} | 23.0 ± 0.2 | 22.2 ± 0.2 | 18.3 ± 0.7 | 3.8 ± <0.1 | 8.5 ± 0.3 | 63.6 ± 1.0 | 15.9 ± 0.2 | 56.6 ± 2.0 |

The series 2 VE-ACM preparations showed that the hydration of ACM prepared within an hour through vacuum evaporation (2.94±0.17 g water/g micellar casein) was very similar to ACM prepared with the Schmidt method (S-ACM; 2.92±0.03 g water/g micellar casein), and that the hydration was not significantly influenced by the preparation time. Meanwhile, the hydration of the ACM prepared via reverse osmosis (RO-ACM) was also very similar to ACM prepared with the Schmidt method (S-ACM), and the ACM prepared via forward osmosis (FO-ACM) was more hydrated compared to S-ACM, and did not appear to be influenced by the preparation time (Table 14):

TABLE 14
Hydration of the prepared ACM.

| | Hydration (g water/g micellar casein) |
|---|---|
| S-ACM_{t=60 min} | 2.92 ± 0.03 |
| VE-ACM_{t=41 min} | 2.84 ± 0.01 |
| VE-ACM_{t=46 min} | 2.99 ± <0.01 |
| VE-ACM_{t=60 min} | 2.94 ± 0.17 |
| VE-ACM_{t=80 min} | 2.90 ± 0.01 |
| VE-ACM_{t=105 min} | 3.02 ± 0.01 |
| FO-ACM_{t=28 min} | 4.2 ± <0.1 |
| FO-ACM_{t=38 min} | 4.4 ± 0.1 |
| FO-ACM_{t=60 min} | 3.8 ± 0.4 |
| RO-ACM_{t=22 h} | 3.1 ± <0.1 |

Rennet coagulation of ACM

The ACM produced in the Series 1 vacuum evaporation experiments had different coagulation properties, which were evaluated by in situ rheological characterization. The maximum storage modulus (G′) represents the firmness of the formed curd within an hour of renneting.

The maximum G′ of samples prepared with different concentration factors is presented in FIG. 7a. Skim milk, which was diluted to the same casein concentration of ACM, attained a maximum G′ of 117.3 Pa within an hour of incubation with rennet. Sedimentation prior to vacuum evaporation was observed for the samples evaporated by 3× and 4.3× concentration, resulting in weaker rennet coagulation as evident by their maximum G′ of 65.7 and 85.7 Pa, respectively. The sample concentrated 10× showed the highest G′ with 154.6±6.2 Pa, whereas the sample concentrated 6× attained a slightly lower maximum G′ of 140.6±<0.1 Pa. The ACM concentrated 30× was the closest to the skim milk reference with 118.4±<0.1 Pa. For comparison, ACM prepared by the method of Schmidt coagulated with a maximum G′ of 122.5 Pa.

The maximum G′ for samples prepared with different evaporation rates is shown in FIG. 7b. The micelles formed at different evaporation rates coagulated with a maximum G′ of 166.4±0.8 Pa for the ACM evaporated at 373 mL h⁻¹. The sample with the lowest G′ was evaporated at 655 mL h⁻¹ and attained a G′ of 133.1 Pa. ACM prepared according to the Schmidt method coagulated with a maximum G′ of 122.5 Pa, which is very close to the reference sample of skim milk with 117.6±0.4 Pa. Overall, the ACM produced via vacuum evaporation showed higher maximum storage moduli (except when prepared with low concentration factors) than the ACM produced by the method of Schmidt and those in skim milk.

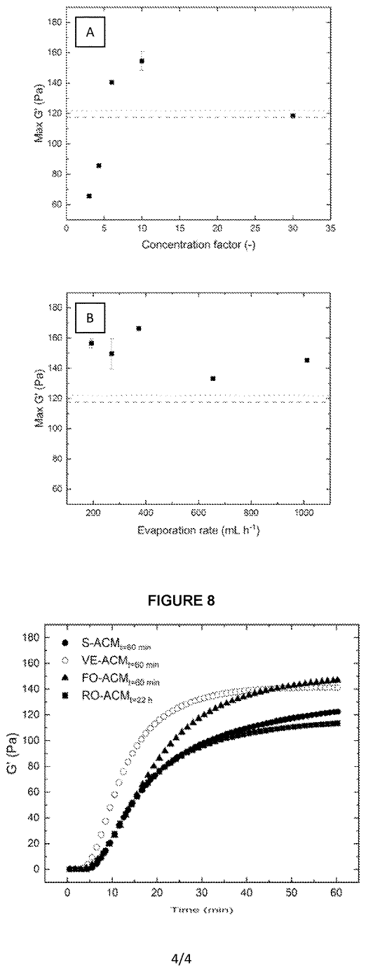

The VE-ACM from the Series 2 vacuum evaporation experiments were also compared based on their coagulation behaviour upon renneting, using the same protocols as those applied above for the Series 1 VE-ACM. ACM prepared through vacuum evaporation had a faster onset of coagulation and formed a firmer coagulum compared to ACM prepared by the method of Schmidt (141.6±5.4 Pa versus 122.6±1.0 Pa; Table 15) within an hour of incubation with rennet (FIG. 8). Forward osmosis preparation of ACMs (FO-ACM) produced equally firm curds as vacuum evaporation, but the preparation time of FO-ACM did not influence curd firmness. Reverse osmosis preparation of ACMs (RO-ACM) produced curds of similar firmness as ACM prepared with the Schmidt method since the RO-ACM contained lower concentrations of casein and minerals. A comparison of all four preparation methods is shown in FIG. 8. Increasing preparation times generally has a positive effect on the resulting curd firmness (Table 15):

TABLE 15
Maximum storage modulus G′ of the prepared samples
attained during one hour of incubation with rennet.

| | Max G′ (Pa) |
|---|---|
| S-ACM_{t=60 min} | 122.6 ± 1.0 |
| VE-ACM_{t=105 min} | 157.9 ± 0.2 |
| VE-ACM_{t=80 min} | 138.9 ± 0.2 |
| VE-ACM_{t=60 min} | 141.6 ± 5.4 |
| VE-ACM_{t=46 min} | 144.2 ± 2.8 |
| VE-ACM_{t=41 min} | 117.3 ± 1.1 |
| FO-ACM_{t=28 min} | 140.5 ± 1.5 |
| FO-ACM_{t=38 min} | 132.3 ± 3.3 |
| FO-ACM_{t=60 min} | 147.0 ± 12.2 |
| RO-ACM_{t=22 h} | 113.6 ± 0.1 |

### Conclusions

The disclosure herein provides a process for assembling non-micellar caseins into artificial casein micelles that is more well suited to scale-up than existing approaches such as the prior art mixing method of Schmidt, 1 for at least the reason that it overcomes many of the problems associated with local excesses of titrants leading to local ionic excesses allowing the potential for immediate clustering of ions, arising from drops of concentrated salt solutions in areas of high salt concentration being present before stirring can distribute the ions evenly, with the result that the caseins do not have to align themselves in ideal positions as rapidly as during the prior art mixing methods.

Further advantages in the process of the present invention arise from the observation that increasing preparation times has a positive effect on the resulting curd firmness after renneting, providing the ability to tune the process to achieve the desired curd firmness for the particular downstream product (e.g.; cheese type) to be produced.

ACM prepared via the process of the present invention are similar to ACM prepared through the Schmidt method under the same conditions (temperature and preparation time). ACM prepared via the process of the present invention contain a larger proportion of micellar casein, are slightly less mineralised, coagulate faster and form firmer curds, but are similar in hydration and size to ACM prepared through the Schmidt method. The preparation time did not influence the proportion of micellar casein, the mineralisation and the hydration of ACM, but did have an influence on the micelle size and, desirably, on the maximum curd firmness upon renneting.

### General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

It should be appreciated that throughout this specification, any reference to any prior publication, including prior patent publications and non-patent publications, is not an acknowledgment or admission that any of the material contained within the prior publication referred to was part of the common general knowledge as at the priority date of the application.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. size, displacement and field strength etc). A range of values will be understood to include all values within the range, including the values defining the range, and values adjacent to the range which lead to the same or substantially the same outcome as the values immediately adjacent to that value which defines the boundary to the range.

The present invention is not to be limited in scope by any of the specific embodiments described herein. These embodiments are intended for the purpose of exemplification only. Functionally equivalent products, formulations and methods are clearly within the scope of the invention as described herein.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

### REFERENCES

1. Schmidt, D. G., Koops, J., & Westerbeek, D. (1977). Properties of artificial casein micelles. 1. Preparation, size distribution and composition. Netherlands Milk and Dairy Journal, 31(4), 328-341.
2. Gaucheron, F. (2005). The minerals of milk. Reproduction Nutrition Development, 45(4), 473-483.
3. Dumpler, J., Kieferle, I., Wohlschläger, H., & Kulozik, U. (2017). Milk ultrafiltrate analysis by ion chromatography and calcium activity for SMUF preparation for different scientific purposes and prediction of its supersaturation. International Dairy Journal, 68, 60-69. https://doi.org/10.1016/j.idairyj.2016.12.009.
4. Huppertz, T., Gazi, I., Luyten, H., Nieuwenhuijse, H., Alting, A., & Schokker, E. (2017). Hydration of casein micelles and caseinates: Implications for casein micelle structure. International Dairy Journal, 74, 1-11. https://doi.org/10.1016/j.idairyj.2017.03.006
5. Schubert, T., Meric, A., Boom, R., Hinrichs, J., & Atamer, Z. (2018). Application of a decanter centrifuge for casein fractionation on pilot scale: Effect of operational parameters on total solid, purity and yield in solid discharge. International Dairy Journal, 84, 6-14.
6. Guimarães, B. O., Gremmen, P., Wijffels, R. H., Barbosa, M. J., & D'Adamo, S. (2021). Effect of ammonium formate washing on the elemental composition determination in Nannochloropsis oceanica. Aquaculture, 538, 736526. https://doi.org/10.1016/j.aquaculture.2021.736526.
7. Pierre, A., & Brule, G. (1981). Mineral and protein equilibria between the colloidal and soluble phases of milk at low temperature. Journal of Dairy Research, 48(3), 417-428.
8. Bijl, E., van Valenberg, H. J. F., Huppertz, T., & van Hooijdonk, A. C. M. (2013). Protein, casein, and micellar salts in milk: Current content and historical perspectives. Journal of Dairy Science, 96(9), 5455-5464.
9. Antuma, L. J., Braitmaier, S. H., Garamus, V. M., Hinrichs, J., Boom, R. M., & Keppler, J. K. (2024). Engineering artificial casein micelles for future food: Preparation rate and coagulation properties. Journal of Food Engineering, 366, 111868. https://doi.org/10.1016/j.jfoodeng.2023.111868

1. A process for the preparation of Artificial Casein Micelles (ACMs), comprising the steps of:
(a) preparing a solution comprising non-micellar caseins and calcium phosphate, wherein the concentration of calcium phosphate is dilute; and
(b) concentrating the solution comprising non-micellar caseins and calcium phosphate to form a solution comprising ACMs;
wherein the formation of the ACMs is induced by step (b) concentrating the solution comprising non-micellar caseins and calcium phosphate, without the addition of any further salts or caseins to the solution prepared in step (a).

2. The process of claim 1, wherein step (b) comprises removal of solvent by evaporation under reduced pressure, membrane processes, forward osmosis, or reverse osmosis.

3. The process of claim 1, wherein step (b) further comprises controlling the pH of the solution while concentrating the solution by the addition of a suitable base, to maintain the pH of the solution at a pH of pH 5 to pH 7.5, pH 6 to pH 7, or a pH of 6.7; optionally wherein the base is sodium hydroxide.

4. The process of claim 1, wherein the solution prepared in step (a) further comprises one or more additional species selected from the group consisting of calcium, magnesium, sodium, potassium, chloride, phosphate, phosphorus, citrate, carbonate, sulfate, nitrate, hydroxide, and lactate.

5. The process of claim 1, wherein the solution comprising ACMs formed in step (b) has a pH of pH 5 to pH 7, pH 6 to pH 7, or a pH of 6.7.

6. The process of claim 1, wherein the solution prepared in step (a) has a pH, prior to commencement of step (b), of pH 6 to pH 8, pH 6.2 to pH 7.8, or pH 6.5 to 7.5.

7. The process of claim 1, wherein the non-micellar caseins are one or more non-micellar caseins selected from the group consisting of αs1-casein, αs2-casein, β-casein, and κ-casein.

8. The process of claim 1, wherein the non-micellar caseins are isolated from mammalian milk, or wherein the non-micellar caseins are recombinantly produced non-micellar caseins.

9. The process of claim 1, wherein the non-micellar caseins are phosphorylated or not phosphorylated; and/or wherein the non-micellar caseins are glycosylated or not glycosylated.

10. The process of claim 1, wherein the solution comprising ACMs formed in step (b) comprises:
calcium at a concentration of 20 mmol/kg to 40 mmol/kg, 25 mmol/kg to 35 mmol/kg, or 26 mmol/kg to 31 mmol/kg; and/or
magnesium at a concentration of 2 mmol/kg to 8 mmol/kg or 4 mmol/kg to 6 mmol/kg; and/or
inorganic phosphate at a concentration of 10 mmol/kg to 30 mmol/kg, 15 mmol/kg to 25 mmol/kg, or 19 mmol/kg to 23 mmol/kg; and/or
total phosphorus at a concentration of 20 mmol/kg to 40 mmol/kg, 25 mmol/kg to 35 mmol/kg, or 26 mmol/kg to 32 mmol/kg; and/or
citrate at a concentration of 2 mmol/kg to 20 mmol/kg or 7 mmol/kg to 11 mmol/kg; and/or
sodium at a concentration of 5 mmol/kg to 100 mmol/kg, 10 mmol/kg to 80 mmol/kg, or 15 mmol/kg to 75 mmol/kg; and/or
potassium at a concentration of 10 mmol/kg to 50 mmol/kg or 15 mmol/kg to 40 mmol/kg; and/or
chloride at a concentration of 5 mmol/kg to 100 mmol/kg, 10 mmol/kg to 80 mmol/kg, or 15 mmol/kg to 75 mmol/kg.

11. The process of claim 1, wherein the solution prepared in step (a) comprises Mg²⁺, PO₄³⁻, Ca²⁺, citrate, and non-micellar caseins in the ratios in which they occur in bovine milk.

12. The process of claim 1, wherein the solution comprising ACMs formed in step (b) comprises total caseins at a concentration of 15 g/L to 100 g/L or 20 g/L to 30 g/L.

13. The process of claim 1, wherein the solution comprising ACMs formed in step (b) comprises ACMs with a Z-average diameter of 40 nm to 500 nm.

14. The process of claim 1, wherein the solution comprising ACMs formed in step (b) comprises ACMs with hydration values of 2 to 4, wherein said hydration value is g water/g micellar casein.

15. The process of claim 1, further comprising the step (c) coagulating the solution comprising ACMs formed in step (b) to form a curds composition.

16. The process of claim 15 wherein step (c) comprises addition of an acid, and/or microbial acidification, and/or addition of a renneting agent.

17. The process of claim 15, wherein the curds composition has a Maximum G′ storage modulus of 50 Pa to 400 Pa, optionally after 1 hour of incubation with a renneting agent.

18. The process of claim 15, further comprising the step of (d) aging and/or maturing the curds composition, to form a cheese composition.

19. The process of claim 1, wherein the process does not comprise any animal-derived protein.

20. The process of claim 1, wherein the process is conducted on an industrial scale; and/or wherein the process is conducted continuously; and/or wherein the process is conducted on a scale capable of producing 100 L to 10,000 L of the solution comprising ACMs formed in step (b) in a single batch or process run.

21. The process of claim 1, wherein the solution prepared in step (a) is prepared with a Concentration factor of 3× to 50× or 6× to 30×.

22. The process of claim 1, wherein step (b) comprises removal of solvent by evaporation at a reduced pressure of 10 mbar to 300 mbar, corresponding to a boiling point of water falling within the range of 7° C. to 70° C.

23. The process of claim 1, wherein the solution prepared in step (a) comprises calcium at a concentration of 0.6 mM to 10 mM; and/or phosphate at a concentration of 0.4 mM to 7.3 mM.
